(19) United States
(12) Patent Application Publication
(10) Pub. No.: US 2004/0237128 A1
(43) Pub. Date: Nov. 25, 2004

(54) Transgenic non-human animal having a disruption of at least one allele to the ceacam 1 gene and method of making same

(76) Inventors: Nicole Beauchemin, Montreal (CA); Claire Turbide, Montreal (CA); Kathryn V Holmes, Golden, CO (US); Dianna Blau, Jacksonville, FL (US); Stephanie Letourneau, Montreal (CA)

Correspondence Address: GOUDREAU GAGE DUBUC, 800 PLACE VICTORIA, SUITE 3400, MONTREAL, QUEBEC, H4Z 1E9, CA

(21) Appl. No.: 10/473,424

(22) PCT Filed: Apr. 4, 2002
(86) PCT No.: PCT/CA02/00469

Related U.S. Application Data
(60) Provisional application No. 60/281,025, filed on Apr. 4, 2001.

(51) Int. Cl. A01K 67/027

(52) U.S. Cl. 800/18

(57) ABSTRACT

The present invention relates to a transgenic animal having a disruption of at least one allele of the Ceacam1 gene and method of making same. More particularly, the present invention relates to transgenic mouse whose germ cells and somatic cells contain a knockout mutation in DNA encoding CEACAM1^{a} or CEACAM1^{b}. In a particular embodiment, mice containing a disruption of at least one of the Ceacam1 gene present a reduced expression of the CEACAM1^{a} protein. The present invention also relates to a transgenic mouse comprising a disruption of the Ceacam1 gene wherein the mouse expresses at least a reduced level of CEACAM1^{a} relative to a corresponding wild-type mouse and methods of making same.

### FIELD OF THE INVENTION

The present invention relates to a transgenic non human animal having a disruption of at least one allele of the CEACAM1 gene and method of making same. More particularly the present invention relates to a transgenic mouse whose germ cells and somatic cells contain a knockout mutation in DNA encoding CEACAM1. The present invention also relates to methods of making the transgenic animal and in particular a transgenic mouse whose germ cells and somatic cells contain a knockout mutation in the Ceacam1 gene and to vectors comprising this knockout mutation.

### BACKGROUND OF THE INVENTION

The CEACAM1 glycoprotein (formerly called biliary glycoproteins, BGP, Bpg, C-CAM, mmCGM1, CD66a or MHVR) is a member of the carcinoembryonic antigen (CEA) family of molecules (2, 14, 25). The nomenclature for the CEA gene family has recently been unified (6), and 7 genes are now referred to as the CEACAM genes. CEACAM1, the most conserved gene of the CEA gene family, is found as a single copy on human chromosome 19q13.2 (24) and in the rat genome. Three highly homologous genes were located on mouse chromosome 7 in the 7A2-A3 region near the centromere, a region syntenic to human chromosome 19q, identified as the Ceacam1, Ceacam2 (formerly Bgp2) and Ceacam10 genes, respectively (23, 24,). The murine Ceacam1 and Ceacam2 genes are positioned one next to the other on the chromosome and share a high degree of homology in their exon-intron as well as in their nucleotide sequences (23, 24). The Ceacam10 gene is different in many of its features and positioned approximately 0.7 cM distal to the Ceacam1 and Ceacam2 genes. The human, rat and mouse Ceacam1 genes have highly conserved gene structures (2, 23), 66% identity between their respective promoters (23), similar but not identical patterns of mRNA splicing and similar patterns of expression in different tissues (15).

The CEACAM1 glycoprotein isoforms are normally expressed at the surface of epithelial cells, endothelial and hemopoietic cells (5). They are heavily glycosylated proteins with as many as 16 N-linked sugar chains attached to the extracytoplasmic Ig domains.

The CEACAM1 protein serves as the receptor for a number of pathogens, including murine coronavirus (MHV), Hemophilus influenzae and binds to the opa surface proteins of various pathogenic strains of Neisseria (gonorrhea, meningitidis, etc) in humans. In addition to MHV, a murine coronavirus, there are coronaviruses specific for other species including humans, pigs, dogs, cats, cattle, rats, birds, etc. None of these other viruses causes hepatitis as MHV does in its natural host. Hepatitis in these animals is caused by different families of viruses that use different cellular molecules as receptors for virus entry. The CEACAM1 protein has cell adhesion activity and acts as a signaling molecule (25). It was recently reported that CEACAM1 plays a role in the down-regulation of the cytolytic functions of activated human intestinal intraepithelial lymphocytes and that it behaves as an angiogenic factor. Certain CEACAM1 isoforms inhibit the development of colon and prostate tumors in mouse and rat models (18). In addition, levels of CEACAM1 expression are reduced early in the development of human cancers of the colon, prostate, liver, bladder, endometrium and breast.

Mouse CEACAM1 isoforms are transmembrane glycoproteins that have either two or four immunoglobulin (Ig) domains produced by alternative splicing of the primary transcript (20). This will be presented in more details below. CEACAM1 isoforms with four Ig-like domains, denoted as CEACAM1/D1-4, include the N domain (D1) attached to three constant Ig-like domains (D2, D3 and D4). Splice isoforms with 2 Ig-like domains (CEACAM1/D1,4) link the N domain to the fourth Ig-like domain. The exodomains are linked to a transmembrane domain followed by either of two cytoplasmic tails. The short cytoplasmic tail (CEACAM1-S) contains 10 amino acids rich in Ser and Gly residues. The long cytoplasmic tail (CEACAM1-L) results from inclusion of the 53 bp exon 7 that shifts the open reading frame of the tail at aa 453 and yields a 73 aa tail (20, 24).

The CEACAM1 proteins are abundantly expressed in epithelial cells along the gastrointestinal and respiratory tracts, in bile canaliculi and on the proximal tubules of the kidney. They are also found on small vascular endothelial cells, in hemopoietic cells (B cells, neutrophils, macrophages, monocytes, platelets, and activated T cells) (10, 15). They are expressed at the surface of epithelial cells in the reproductive tissues (uterus, ovary, breast and prostate) (18) and on glial cells in the nervous system (15). CEACAM1 is abundantly expressed during mouse embryonic development in endodermal and mesenchymal derivatives.

The mouse hepatitis virus (MHV) is a murine coronavirus that causes respiratory and enteric infections, hepatitis, splenolysis, immune dysfunction, acute encephalitis and chronic demyelinating disease of the brain and spinal cord (3). MHV infection of mice can be inapparent and self-limited or can lead to chronic and systemic infection and death. In some mouse strains, non-apparent MHV infections can result in disruption of normal cytokine patterns for at least 5 months after infection. Many but not all of the tissues expressing CEACAM1 are natural targets for MHV infection (3, 4, 17).

Williams et al., (1990) and Dveksler et al., (1991) identified and cloned the cDNA encoding the CEACAM1^{a}/D1-4 MHV receptor (12). The sequence of the virus receptor was found to be identical to the biliary glycoprotein identified as a CEA-related cell adhesion glycoprotein. Adult SJL mice that are highly resistant to MHV infections (4) are homozygous for CEACAM1^{b}, an allele of CEACAM1^{a}, that differs by 27 of 108 aa in the N domain (D1) (14, 32). The viral spike glycoprotein (S) attaches to the N domain of CEACAM1^{a}(14). Most inbred strains of mice (BALB/c, C57BI/6, C3H, 129Sv, FVB) are susceptible to MHV infection and express the CEACAM1^{a} allele. Outbred CD1 mice express both CEACAM1^{a} and CEACAM1^{b} alleles. When the murine Ceacam1^{a}/D1-4, short tail cDNA was transfected into hamster cell lines that are not susceptible to MHV, expression of the mouse CEACAM1^{a} glycoprotein rendered the cells susceptible to MHV (12). All MHV strains tested to date utilize the murine CEACAM1 proteins as receptors (13). Mutational analyses showed that the virus binds to the B-C-C′ region of the first immunoglobulin (Ig) domain D1 of the CEACAM1^{a} receptor (14, 28, 32). A monoclonal antibody (Mab CC1) directed against the first domain of CEACAM1^{a} blocks virus attachment and prevents infections in vitro and in vivo (30). Infant mice were treated intraperitoneally and intranasally with anti-CEACAM1^{a} monoclonal antibody CC1 and this treatment protected the mice from death due to MHV inoculation. This experiment was only successful on mouse younger than three weeks old however (30). CEACAM1^{a} isoforms with either 2 or 4 Ig-like domains (expressing either the short or long tail) serve as receptors for MHVs when expressed at high levels in hamster cells (13). CEACAM2, the product of a different but highly homologous gene (Ceacam2) also serves as a MHV receptor, although it is a markedly less efficient MHV-A59 receptor, as compared to CEACAM1^{a} (23, 33). Of note, expressing high levels of CEACAM1^{b} in hamster cells also renders cells susceptible to viral infections (13).

The surface density of CEACAM1^{a} affects susceptibility to infection with the MHV-JHM viral strain. Hela cells induced to express low amounts of recombinant murine CEACAM1^{a} were susceptible to infections by MHV-JHM, but did not exhibit cytopathic effects such as cell fusion and death. In contrast, Hela cells induced to express high levels of CEACAM1^{a} on the cell surface died within 14 h of infection with MHV-JHM (29). Complexes between the CEACAM1^{a} receptor and the viral S spike glycoprotein formed in the ER and Golgi. Expression of S glycoprotein or MHV infection of murine cells leads rapidly to selection of cells that express reduced levels of CEACAM1^{a}. However, persistent infection with the MHV-A59 virus leads to selection of cells resistant to wild-type and selection of mutant viruses that form small plaques and have mutations in the R binding domain of the viral S glycoprotein (16).

Taken together, the functions of CEACAM1 have therefore so far been examined only using in vitro model systems based on cell biology.

To further elucidate the mechanisms responsible for the many biological activities of CEACAM1, there thus remains a need to study the expression of the Ceacam1 gene modified in a more physiological context. In addition, there remains a need to provide mice that are resistant to MHV strains and/or other infections agents which utilize CEACAM1 as a receptor The present invention seeks to meet these and other needs.

The present description refers to a number of documents, the content of which is herein incorporated by reference in their entirety.

### SUMMARY OF THE INVENTION

The invention concerns a non-human transgenic animal having a disruption of at least one allele of the CEACAM1 gene. In a particular embodiment this disruption renders the non-human transgenic animal partially or totally resistant to an infectious agent (e.g. pathogen) which utilizes CEACAM1 as a receptor. In a particularly preferred embodiment, this infectious agent is a virus or a bacteria.

The present invention therefore provides non-human transgenic animals having at least one allele of CEACAM1 disrupted. More particularly, the present invention relates to transgenic mice having at least one allele of CEACAM1 disrupted. Experiments with these have yet to reveal significant differences between same and wild type mice. It remains to be determined whether under specific conditions, the knock-out mice are histologically, biochemically or physiologically different than their wild-type counterparts.

The invention also concerns a transgenic mouse having a disruption of at least one allele of the Ceacam1 gene that is partially or totally resistant to MHV, a murine coronavirus that uses CEACAM1 isoforms as receptors. The invention also concerns methods of modulating MHV infection sensitivity comprising a modulation of the alternative splicing of the Ceacam1 gene.

The invention further concerns a non-human transgenic animal having a disruption of at least one allele of the CEACAM1 gene as an animal model for the study of diseases or conditions associated with a function or lack thereof of a CEACAM1 receptor or part thereof. In a particular embodiment, this non-human transgenic animal has both copies of the CEACAM1 gene disrupted.

The applicant is the first to provide an animal model for the study of the modulation of MHV infection, a Ceacam1^{a}-mutated mice having a reduced level of expression of the CEACAM1^{a} virus receptor glycoproteins in tissues that serve as targets for infectious agent. In a preferred embodiment, the mice are Ceacam1^{a}-targeted (Ceacam1^{a}Δ4D) mice.

The applicant is also the first to provide a viable and fertile Ceacam1 disrupted mice having no gross tissue abnormalities.

The applicant is the first to show that a modulation of the level of CEACAM1 splice isoforms in vivo can result in targeted mice with 1 significantly decreased susceptibility to MHV infection.

As seen above, the role of CEACAM1^{a} glycoprotein in MHV susceptibility was known in hamster cell lines that, while initially not susceptible to MHV, were rendered susceptible to this virus after being transfected by mouse CEACAM1^{a} glycoprotein. Further, it was shown that infant mice treated with anti-CEACAM1^{a} monoclonal antibody CC1 were rendered resistant to MHV infection. However, prior to the present invention, no experimental result existed showing how a knock out of the Ceacam1^{a} gene in a mouse would affect its susceptibility to MHV.

Transfection results obtained with a cell line are insufficient to reasonably predict the outcome of a transfection of this protein in a living animal. Indeed, several mice transgenic models for a human receptor protein for a human virus have generated mice that are not susceptible to the human virus. An unrecognized co-receptor might be required for susceptibility to arise, and other host factors may inactivate the virus. In addition, the level of receptor expression specific to target tissues for infection, have to be carefully controlled to sustain an infection.

Similarly, results obtained by inactivating a receptor in a mouse by a monoclonal antibody directed against same are insufficient to predict the outcome of a knock out of this receptor in a mouse. Prior to the present invention, it was impossible to predict whether other co-factors responsible for the virus susceptibility would not compensate for the decrease in the receptor expression. Further, it was impossible to predict how the knock out of a receptor would affect other functions in the mouse (or in the animal model used). Thus, prior to the present invention, there was no way to predict with certainty what strategy would successfully generate a susceptible transgenic or a resistant knockout.

In accordance with one embodiment of the present invention, there is therefore provided a transgenic mouse comprising a disruption of the Ceacam1 gene wherein the mouse expresses a reduced level of CEACAM1 relative to a corresponding wild-type mouse.

In accordance with another embodiment of the present invention, there is also provided a method for producing a transgenic mouse wherein the mouse expresses a reduced level of CEACAM1 relative to a corresponding wild-type mouse, comprising introducing a CEACAM1 targeting vector into a mouse embryonic stem cell; introducing the mouse embryonic stem cell into a mouse blastocyst; transplanting the non-human animal blastocyst into a pseudopregnant non-human animal; allowing the non-human animal blastocyst to develop to term; identifying a transgenic mouse whose genome comprises a disruption of the Ceacam1 gene in at least one allele; breeding the thereby obtained transgenic mouse to obtain a transgenic mouse whose genome comprises a disruption of the Ceacam1, wherein the disruption results in decreased levels of CEACAM1 relative to a wild-type mouse.

In accordance with yet another embodiment of the present invention, there is provided a method of modulating an infection by an infectious agent which uses a region, or epitope of CEACAM1 as a receptor, which comprises a modulation of the expression of at least one isoform of CEACAM1. In a particularly preferred embodiment, there is provided a method of modulating MHV infection sensitivity in a cell and/or in a mouse comprising a modulation of the expression of Ceacam1. In a particular embodiment, the ratio of 4 Ig isoforms of CEACAM1^{a} over 2 Ig isoforms of CEACAM1^{a} is reduced relative to a corresponding ratio in a wild-type cell and/or mouse.

Definitions

The present description refers to a number of routinely used terms, including recombinant DNA (rDNA) technology terms. Nevertheless, definitions of selected examples of such terms are provided for clarity and consistency.

As used herein, the terminology “transgenic animal” refers to any animal which harbors a nucleic acid sequence having been inserted into a cell and having become part of the genome of the animal that develops from that cell. In a preferred embodiment, the transgenic animal is a mouse. Techniques for the preparation of such transgenic animals are well known in the art (e.g. introducing a transgene in embryonic stem (ES) cells; microinjecting the modified ES cells into blastocyst; or infecting a cell with a recombinant virus containing the transgene in its genome). Non-limiting examples of patents relating to a non-human transgenic animal include U.S. Pat. Nos. 4,736,866; 5,087,571; 5,175,383; 5,175,384 and 5,175,385.

As used herein, “transgenic mouse” relates to any mouse in which at least one cell comprises genetically altered information through known means such as microinjection, virus-delivered infection, or homologous recombination. In one particularly preferred embodiment of the present invention, the genetic alteration of the transgenic mouse has been introduced in a germ-line cell, such that it enables the transfer of this genetic alteration to the offsprings thereof. Such offsprings, containing this generic alteration are also transgenic mice.

The terminology “gene knockout” or “knockout” refers to a disruption of a nucleic acid sequence that significantly reduces and preferably suppresses or destroys the biological activity of the polypeptide encoded thereby. For example, a CEACAM1^{a} knockout mouse refers to a mouse in which the expression of CEACAM1^{a} and/or CEACAM1^{b} has been reduced or suppressed by the introduction of a recombinant nucleic acid molecule comprising CEACAM1^{a} and/or CEACAM1^{b} sequences that disrupt at least a portion of the genomic DNA sequence encoding CEACAM1^{a} and/or CEACAM1^{b} therein. A knockout mouse might have one or both copies of the pre-selected nucleic acid sequence disrupted. In the latter case, in which a homozygous disruption is present, the mutation is termed a “null” mutation. In a case where only one copy of a pre-selected nucleic acid sequence is disrupted, the knockout mouse is a “heterozygous knockout mouse”. In a preferred embodiment CEACAM1^{a} is disrupted.

While the transgenic mice of the present invention are exemplified with MHV, E. coli and Salmonella infections, the invention should not be so limited. Indeed, the present invention provides in one embodiment transgenic animals and particularly mice that have an elevated resistance to a virus or bacteria which uses CEACAM1 as a receptor by virtue of a disruption of at least one CEACAM1 allele, as compared to a control animal not having such a disruption. It will be understood that the present invention is meant to cover a non-human transgenic animal resistant to a pathogen by virtue of the knocking-out of the domain of CEACAM1 responsible for pathogen entry into the cell.

The term “fragment”, as applied herein to a peptide, refers to at least 7 contiguous amino acids, preferably about 14 to 16 contiguous amino acids, and more preferably, more than 40 contiguous amino acids in length. Such peptides can be produced by well-known methods to those skilled in the art, such as, for example, by proteolytic cleavage, genetic engineering or chemical synthesis.

The terminology “modulation of two factors” is meant to refer to a change in the affinity, strength, rate and the like between such two factors. The terminology “modulation of translation” refers to change in the efficiency or rate of translation of mRNAs resulting in a quantitative or qualitative change or rate of protein synthesis.

Nucleotide sequences are presented herein by single strand, in the 5′ to 3′ direction, from left to right, using the one letter nucleotide symbols as commonly used in the art and in accordance with the recommendations of the IUPAC-IUB Biochemical Nomenclature Commission.

Unless defined otherwise, the scientific and technological terms and nomenclature used herein have the same meaning as commonly understood by a person of ordinary skill to which this invention pertains. Generally, the procedures for cell cultures, infection, molecular biology methods and the like are common methods used in the art. Such standard techniques can be found in reference manuals such as for example Sambrook et al. (1989, Molecular Cloning-A Laboratory Manual, Cold Spring Harbor Laboratories) and Ausubel et al. (1994, Current protocols in Molecular Biology, Wiley, New York).

As used herein, “nucleic acid molecule”, refers to a polymer of nucleotides. Non-limiting examples thereof include DNA (e.g. genomic DNA, cDNA), RNA molecules (e.g. mRNA) and chimeras thereof. The nucleic acid molecule can be obtained by cloning techniques or synthesized. DNA can be double-stranded or single-stranded (coding strand or non-coding strand [antisense]).

The term “recombinant DNA” as known in the art refers to a DNA molecule resulting from the joining of DNA segments. This is often referred to as genetic engineering.

The term “DNA segment” is used herein to refer to a DNA molecule comprising a linear stretch or sequence of nucleotides. This sequence when read in accordance with the genetic code, can encode a linear stretch or sequence of amino acids which can be referred to as a polypeptide, protein, protein fragment and the like.

As used herein, “oligonucleotides” or “oligos” define a molecule having two or more nucleotides (ribo or deoxyribonucleotides). The size of the oligo will be dictated by the particular situation and ultimately on the particular use thereof and adapted accordingly by the person of ordinary skill. An oligonucleotide can be synthetised chemically or derived by cloning according to well known methods.

The nucleic acid (e.g. DNA or RNA) for practicing the present invention may be obtained according to well known methods.

The term “DNA” molecule or sequence refers to a molecule generally comprised of the deoxyribonucleotides adenine (A), guanine (G), thymine (T) and/or cytosine (C), which in a double-stranded form, can comprise or include a “regulatory element” according to the present invention, as the term is defined herein. “DNA” can be found in linear DNA molecules or fragments, viruses, plasmids, vectors, chromosomes or synthetically derived DNA. As used herein, particular double-stranded DNA sequences may be described according to the normal convention of giving only the sequence in the 5′ to 3′ direction. The same applies to single-stranded DNA sequences. As well known in the art, DNA can also be found as circular molecules.

“Nucleic acid hybridization” refers generally to the hybridization of two single-stranded nucleic acid molecules having complementary base sequences, which under appropriate conditions will form a thermodynamically favored double-stranded structure. Examples of hybridization conditions can be found in the two laboratory manuals referred above (Sambrook et al., 1989, supra and Ausubel et al., 1989, supra) and are commonly known in the art. In the case of a hybridization to a nitrocellulose filter, as for example in the well known Southern blotting procedure, a nitrocellulose filter can be incubated overnight at 65° C. with a labelled probe in a solution containing 50% formamide, high salt (5×SSC or 5× SSPE), 5× Denhardt's solution, 1% SDS, and 100 μg/ml denatured carrier DNA (e.g. salmon sperm DNA). The non-specifically binding probe can then be washed off the filter by several washes in 0.2×SSC/0.1% SDS at a temperature which is selected in view of the desired stringency: room temperature (low stringency), 42° C. (moderate stringency) or 65° C. (high stringency). The selected temperature is based on the melting temperature (Tm) of the DNA hybrid. Of course, RNA-DNA hybrids can also be formed and detected. In such cases, the conditions of hybridization and washing can be adapted according to well known methods by the person of ordinary skill. Stringent conditions will be preferably used (Sambrook et al., 1989, supra).

Probes of the invention can be utilized with naturally occurring sugar-phosphate backbones as well as modified backbones including phosphorothioates, dithionates, alkyl phosphonates and V-nucleotides and the like. Modified sugar-phosphate backbones are generally taught by Miller, 1988, Ann. Reports Med. Chem. 23:295 and Moran et al., 1987, Nucleic acid molecule. Acids Res., 14:5019. Probes of the invention can be constructed of either ribonucleic acid (RNA) or deoxyribonucleic acid (DNA).

The types of detection methods in which probes can be used include Southern blots (DNA detection), dot or slot blots (DNA, RNA), and Northern blots (RNA detection). Although less preferred, labelled proteins could also be used to detect a particular nucleic acid sequence to which it binds. Other detection methods include kits containing probes on a dipstick setup and the like.

Probes can be labelled according to numerous well known methods (Sambrook et al., 1989, supra). Non-limiting examples of labels include ³H, ¹⁴C, ³²P, and ³⁵S. Non-limiting examples of detectable markers include ligands, fluorophores, chemiluminescent agents, enzymes, and antibodies. Other detectable markers for use with probes, which can enable an increase in sensitivity of the method of the invention, include biotin and radionucleotides. It will become evident to the person of ordinary skill that the choice of a particular label dictates the manner in which it is bound to the probe.

As commonly known, radioactive nucleotides can be incorporated into probes of the invention by several methods. Non-limiting examples thereof include kinasing the 5′ ends of the probes using gamma ³²P ATP and polynucleotide kinase, using the Klenow fragment of Pol I of E. coli in the presence of radioactive dNTP (e.g. uniformly labelled DNA probe using random oligonucleotide primers in low-melt gels), using the SP6/T7 system to transcribe a DNA segment in the presence of one or more radioactive NTP, and the like.

As used herein, a “primer” defines an oligonucleotide which is capable of annealing to a target sequence, thereby creating a double stranded region which can serve as an initiation point for DNA synthesis under suitable conditions. In a particularly preferred embodiment, the primer is a single stranded DNA molecule.

Amplification of a selected, or target, nucleic acid sequence may be carried out by a number of suitable methods. See generally Kwoh et al., 1990, Am. Biotechnol. Lab. 8:14-25. Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the Qβ replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra). Preferably, amplification will be carried out using PCR.

Polymerase chain reaction (PCR) is carried out in accordance with known techniques. See, e.g., U.S. Pat. Nos. 4,683,195; 4,683,202; 4,800,159; and 4,965,188 (the disclosures of all three U.S. Patent are incorporated herein by reference). In general, PCR involves, a treatment of a nucleic acid sample (e.g., in the presence of a heat stable DNA polymerase) under hybridizing conditions, with one oligonucleotide primer for each strand of the specific sequence to be detected. An extension product of each primer which is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analyzed to assess whether the sequence or sequences to be detected are present. Detection of the amplified sequence may be carried out by visualization following EtBr staining of the DNA following gel electrophores, or using a detectable label in accordance with known techniques, and the like. For a review on PCR techniques (see PCR Protocols, A Guide to Methods and Amplifications, Michael et al. Eds, Acad. Press, 1990).

Ligase chain reaction (LCR) is carried out in accordance with known techniques (Weiss, 1991, Science 254:1292). Adaptation of the protocol to meet the desired needs can be carried out by a person of ordinary skill. Strand displacement amplification (SDA) is also carried out in accordance with known techniques or adaptations thereof to meet the particular needs (Walker et al., 1992, Proc. Natl. Acad. Sci. USA 89:392-396; and ibid., 1992, Nucleic Acids Res. 20:1691-1696).

As used herein, the term “gene” is well known in the art and relates to a nucleic acid sequence defining a single protein or polypeptide. A “structural gene” defines a DNA sequence which is transcribed into RNA and translated into a protein having a specific amino acid sequence thereby giving rise to a specific polypeptide or protein. It will be readily recognized by the person of ordinary skill, that the nucleic acid sequence of the present invention can be incorporated into anyone of numerous established kit formats which are well known in the art.

A “heterologous” (e.g. a heterologous gene) region of a DNA molecule is a subsegment segment of DNA within a larger segment that is not found in association therewith in nature. The term “heterologous” can be similarly used to define two polypeptidic segments not joined together in nature. Non-limiting examples of heterologous genes include reporter genes such as luciferase, chloramphenicol acetyl transferase, β-galactosidase, and the like which can be juxtaposed or joined to heterologous control regions or to heterologous polypeptides.

The term “vector” is commonly known in the art and defines a plasmid DNA, phage DNA, viral DNA and the like, which can serve as a DNA vehicle into which DNA of the present invention can be cloned. Numerous types of vectors exist and are well known in the art.

The term “expression” defines the process by which a gene is transcribed into one or more mRNAs (transcription), the mRNA is then being translated (translation) into one polypeptide (or protein) or more.

The terminology “expression vector” defines a vector or vehicle as described above but designed to enable the expression of an inserted sequence following transformation into a host. The cloned gene (inserted sequence) is usually placed under the control of control element sequences such as promoter sequences. The placing of a cloned gene under such control sequences is often referred to as being operably linked to control elements or sequences.

Operably linked sequences may also include two segments that are transcribed onto the same RNA transcript. Thus, two sequences, such as a promoter and a “reporter sequence” are operably linked if transcription commencing in the promoter will produce an RNA transcript of the reporter sequence. In order to be “operably linked” it is not necessary that two sequences be immediately adjacent to one another.

Expression control sequences will vary depending on whether the vector is designed to express the operably linked gene in a prokaryotic or eukaryotic host or both (shuttle vectors) and can additionally contain transcriptional elements such as enhancer elements, termination sequences, tissue-specificity elements, and/or translational initiation and termination sites.

Prokaryotic expressions are useful for the preparation of large quantities of the protein encoded by the DNA sequence of interest. This protein can be purified according to standard protocols that take advantage of the intrinsic properties thereof, such as size and charge (e.g. SDS gel electrophoresis, gel filtration, centrifugation, ion exchange chromatography . . . ). In addition, the protein of interest can be purified via affinity chromatography using polyclonal or monoclonal antibodies.

The DNA construct can be a vector comprising a promoter that is operably linked to an oligonucleotide sequence of the present invention, which is in turn, operably linked to a heterologous gene, such as the gene for the luciferase reporter molecule. “Promoter” refers to a DNA regulatory region capable of binding directly or indirectly to RNA polymerase in a cell and initiating transcription of a downstream (3′ direction) coding sequence. For purposes of the present invention, the promoter is bound at its 3′ terminus by the transcription initiation site and extends upstream (5′ direction) to include the minimum number of bases or elements necessary to initiate transcription at levels detectable above background. Within the promoter will be found a transcription initiation site (conveniently defined by mapping with S1 nuclease), as well as protein binding domains (consensus sequences) responsible for the binding of RNA polymerase. Eukaryotic promoters will often, but not always, contain “TATA” boxes and “CCAT” boxes. Prokaryotic promoters contain −10 and −35 consensus sequences, which serve to initiate transcription and the transcript products contain Shine-Dalgarno sequences, which serve as ribosome binding references during translation initiation.

As used herein, the designation “functional derivative” denotes, in the context of a functional derivative of a sequence whether a nucleic acid or amino acid sequence, a molecule that retains a biological activity (either function or structural) that is substantially similar to that of the original sequence (e.g. acting as a receptor for viral infection). This functional derivative or equivalent may be a natural derivative or may be prepared synthetically. Such derivatives include amino acid sequences having substitutions, deletions, or additions of one or more amino acids, provided that the biological activity of the protein is conserved. The same applies to derivatives of nucleic acid sequences which can have substitutions, deletions, or additions of one or more nucleotides, provided that the biological activity of the sequence is generally maintained. When relating to a protein sequence, the substituting amino acid as chemico-physical properties which are similar to that of the substituted amino acid. The similar chemico-physical properties include, similarities in charge, bulkiness, hydrophobicity, hydrophylicity and the like. The term “functional derivatives” is intended to include “fragments”, “segments”, “variants”, “analogs” or “chemical derivatives” of the subject matter of the present invention. It will be understood that in certain mutations (such as exemplified herein), the loss of the biological activity of CEACAM1 (as opposed to the maintenance thereof) to interact with a pathogen, for example, is sought.

As well-known in the art, a conservative mutation or substitution of an amino acid refers to mutation or substitution which maintains: 1) the structure of the backbone of the polypeptide (e.g. a beta sheet or alpha-helical structure); 2) the charge or hydrophobicity of the amino acid; or 3) the bulkiness of the side chain. More specifically, the well-known terminologies “hydrophilic residues” relate to serine or threonine. “Hydrophobic residues” refer to leucine, isoleucine, phenylalanine, valine or alanine. “Positively charged residues” relate to lysine, arginine or hystidine. Negatively charged residues” refer to aspartic acid or glutamic acid. Residues having “bulky side chains” refer to phenylalanine, tryptophan or tyrosine.

The term “variant” refers herein to a protein or nucleic acid molecule which is substantially similar in structure and biological activity to the protein or nucleic acid of the present invention.

The term “allele” defines an alternative form of a gene that occupies a given locus on a chromosome. Non-limiting examples thereof are exemplified with CEACAM1^{a} and CEACAM1^{b}.

As commonly known, a “mutation” is a detectable change in the genetic material which can be transmitted to a daughter cell. As well known, a mutation can be, for example, a detectable change in one or more deoxyribonucleotide. For example, nucleotides can be added, deleted, substituted for, inverted, or transposed to a new position. Spontaneous mutations and experimentally induced mutations exist. The result of a mutations of nucleic acid molecule is a mutant nucleic acid molecule. A mutant polypeptide can be encoded from this mutant nucleic acid molecule.

The present invention also provides antisense nucleic acid molecules that can be used for example to decrease or abrogate the expression of the nucleic acid sequences or proteins of the present invention. An antisense nucleic acid molecule according to the present invention refers to a molecule capable of forming a stable duplex or triplex with a portion of its targeted nucleic acid sequence (DNA or RNA). In one particular embodiment, the antisense is specific to Ceacam1. The use of antisense nucleic acid molecules and the design and modification of such molecules is well known in the art as described for example in WO 96/32966, WO 96/11266, WO 94/15646, WO 93/08845 and U.S. Pat. No. 5,593,974. Antisense nucleic acid molecules according to the present invention can be derived from the nucleic acid sequences and modified in accordance to well known methods. For example, some antisense molecules can be designed to be more resistant to degradation to increase their affinity to their targeted sequence, to affect their transport to chosen cell types or cell compartments, and/or to enhance their lipid solubility by using nucleotide analogs and/or substituting chosen chemical fragments thereof, as commonly known in the art.

It shall be understood that the “in vivo” experimental model can also be used to carry out an “in vitro” assay. For example, cellular extracts from a transgenic animal of the present invention can be prepared and used in one of the in vitro methods of the present invention or an in vitro method known in the art. Such assay could be used to compare the infectious potential of infectious agents on extracts prepared from knock-out versus wild type CEACAM1 animal. Similarly, physiological, biochemical, hematological and/or clinical parameters could be assessed and evaluated between a “knock-out” animal of the present invention versus a “wild-type” animal.

As used herein the recitation “indicator cells” refers to cells that express, in one particular embodiment, the CEACAM1 glycoprotein or domains thereof which interact with a pathogen protein (e.g. viral protein) or other cellular protein co-factors which is directly or indirectly involved in infection by the pathogen, and wherein an interaction between these proteins or interacting domains thereof is coupled to an identifiable or selectable phenotype or characteristic such that it provides an assessment of the interaction between same. Such indicator cells can be used in a screening assay to identify modulators of CEACAM1 function. In certain embodiments, the indicator cells have been engineered so as to express a chosen derivative, fragment, homologue, or mutant of these interacting domains. The cells can be yeast cells or preferably higher eukaryotic cells such as mammalian cells (WO 96/41169). It will be recognized that such indicator cells need not be limited to the study of the interaction between CEACAM1 or part thereof and a protein from a pathogen. Indeed, such indicator cells can be used to evaluate or determine physiological functions of CEACAM1 in a cell or animal.

A host cell or indicator cell has been “transfected” by exogenous or heterologous DNA (e.g. a DNA construct) when such DNA has been introduced inside the cell. The transfecting DNA may or may not be integrated (covalently linked) into chromosomal DNA making up the genome of the cell. In prokaryotes, yeast, and mammalian cells for example, the transfecting DNA may be maintained on a episomal element such as a plasmid. With respect to eukaryotic cells, a stably transfected cell is one in which the transfecting DNA has become integrated into a chromosome so that it is inherited by daughter cells through chromosome replication. This stability is demonstrated by the ability of the eukaryotic cell to establish cell lines or clones comprised of a population of daughter cells containing the transfecting DNA. Transfection methods are well known in the art (Sambrook et al., 1989, supra; Ausubel et al., 1994 supra). In one exemplified embodiment, such an established cell can be derived from a transgenic mouse of the present invention.

### BRIEF DESCRIPTION OF THE DRAWINGS

Having thus generally described the invention, reference will now be made to the accompanying drawings, showing by way of illustration a preferred embodiment thereof, and in which:

FIG. 1, Panel A shows the 9 exons encoded by the mouse Ceacam1^{a} gene; the Ceacam1^{a} allele is distinguished from the Ceacam1^{b} allele vector by nucleotide sequences within exons 2-5. Panel B illustrates the four major alternative splice variants of Ceacam1; Panel C shows the targeting construct that led to the recombination event in ES cells; Panel D shows Southern analyses of genomic DNA from different inbred strains of mice; Panel E illustrates the genotyping of ES cells and mice obtained from recombination events in the ES cells;

FIG. 2 shows the expression of CEACAM1 in various tissues of the non-targeted and targeted mice;

FIG. 3 shows the CEACAM1 immunostaining of tissue sections from wild-type and homozygote mice;

FIG. 4 shows a representative experiment of MHV infections of Ceacam1^{a}Δ4D mice.

FIG. 5 shows the summary of experiments of MHV infections of Ceacam1^{a}Δ4D mice using different MHV viral titers.

FIG. 6 shows the survival of Ceacam1^{a}Δ4D mice, 5 days post infection by salmonella.

FIG. 7 illustrates the genetic organization of the Ceacam1 gene and the constructs used to disrupt both copies thereof. a: The mouse Ceacam1 gene encodes 9 exons. The ATG initiation codon is located in the first exon, whereas two stop codons can be alternatively used, one in exon 8 (TGA(S): used in the translation of isoforms encoding a short cytoplasmic domain) or another in exon 9 (TGA(L): used in the translation of isoforms encoding a long cytoplasmic domain). The dashed lines over and under the gene structure represent the alternative splicing events that produce CEACAM1 isoforms with either two or four Ig domains and either a short or long cytoplasmic domain. b: The Ceacam1 gene produces four major alternatively splice variants. The CEACAM1/D1-4 variants encode the D1 to D4 Ig-like domains (identifed in the boxes) that are linked through a transmembrane domain (TM) to either a short (S) or a long (L) tail. This alternatively spliced event is due to the inclusion of the 53 bp exon 7 (shaded, exon number over the box) resulting in a shift of the open reading frame and the translation of a 73 aa long tail. c: The targeting construct used. The targeting construct that led to a recombination event in ES cells had the TK-neo^{r} selection cassette (blue) inserted into the Xba1-Xho1 sites thereby replacing the first and second exons as well as the first intron of the Ceacam1 gene. d: Structure of the wild-type Ceacam1^{a} allele. Genomic DNA of ES cells or wild-type and heterozygous mice will produce a 12 kb fragment when cleaved with EcoR1. e: Structure of the Ceacam1^{a}-targeted allele. Four probes, identified by the black boxes, were used in Southern analyses. The recombinant allele producing a novel EcoR1 cleaved fragment of 1.7 kb, due to the insertion of the TK-neo^{r}, was detected with either the Nco1 or BamH1-HindIII promoter probes.

FIG. 8 shows Southern analyses of genomic DNA from different ES cell lines and inbred strains of mice following a disruption using the construct of FIG. 7. Mice express three Ceacam-related genes: Ceacam1, Ceacam2 and Ceacam10. Samples of 5 μg of genomic DNA extracted from each ES cell line or mouse strains 2D2 and 11H11 were digested with the EcoR1 restriction enzyme and run on 0.75% agarose gels. The DNA fragments were transferred to Gene Screen Plus and probed with a ³²P-labelled BamH1-HindIII fragment (probe 1 shown in FIG. 7e) located in the proximal promoter region. A characteristic 1.7 kb fragment was apparent in Ceacam1-targeted cells or mice. The wild-type 12 kb EcoR1 fragment was correspondingly reduced in intensity by half (+/−) or eliminated (−/−). Note that the 129Sv/J Ceacam2-specific EcoR1 restriction fragment is shorter than that of other mouse strains.

FIG. 9 shows the expression of Ceacam1^{a} transcripts or proteins. FIG. 9A. To confirm the complete elimination of the Ceacam1^{a} gene transcription, samples of 5 μg of total RNA prepared from either mouse colon or liver tissues of +/+, +/− and −/− mice were separated through formaldehyde-agarose gels and transferred to Hybond N+ membranes. The blots were probed with a ³²P-labelled full-length Ceacam1^{a} cDNA. RNA prepared from wild type BALB/c mouse colon was used as a control (CT). FIG. 9B. Samples of 200:g of colon and liver proteins were separated on SDS-PAGE gels and transferred to Immobilon membranes. Proteins were detected with a polyclonal anti-CEACAM1a antibody (2456) and immune complexes were revealed with ECL detection. FIG. 9C. Blots were cleaned and immunoblotted with a polyclonal anti-actin antibody.

FIG. 10 shows the results of CEACAM1 immunostaining of tissue sections from wild type and −/− homozygous mice. Tissues were immunostained with anti-CEACAM1-specific 2456 polyclonal Ab and lightly counterstained with hematoxylin. FIGS. 10a, b, c and d show sections from the wild type (+/+) mice, whereas e-h are from the −/− mice. FIGS. 10a and e, colon and b and f, ileum: Cross-sections of colonic or intestinal ileum crypts in the −/− mice (e and f) shows no CEACAM1 immunostaining of the luminal membrane as compared to the +/+ mice (a and b). FIGS. 10c and g, kidney: Collecting tubules of the kidney were strongly positive for CEACAM1 in the +/+ mice (g), whereas those of the CEACAM1-targeted mice were negative. FIGS. 10d and h, liver: Hepatocyte bile canaliculi contacts are CEACAM1-positive in the +/+ mice (d), whereas they do not express CEACAM1 in the −/p mice (h). Magnification: 40×.

Other objects, advantages and features of the present invention will become more apparent upon reading of the following non-restrictive description of preferred embodiments with reference to the accompanying drawing which is exemplary and should not be interpreted as limiting the scope of the present invention.

### DESCRIPTION OF THE PREFERRED EMBODIMENT

According to the present invention, a transgenic mouse having its Ceacam1^{a} gene disrupted and exhibiting a reduced or ablated expression of the CEACAM1 protein was obtained. This mouse was obtained by disrupting the mouse Ceacam1^{a} gene in murine 129Sv ES cells, knockout mice were generated and the phenotypes of the resulting mice were examined.

The only mouse that resulted from the first knockout strategy showed only partial reduction in CEACAM1 expression, and showed a markedly altered ratio of splice variants expressed in different murine tissues. Expression of the CEACAM1^{a}D1-4 isoforms was reduced by 90-95% in these mice, whereas the CEACAM1^{a}D1,4 isoforms were increased up to 2 fold. Homozygous Ceacam1^{a}-targeted mice failed to develop clinical signs of viral infection following intranasal inoculation with the MHV-A59 virus which killed wild type mice in 4 days. They developed 50 fold fewer lesions in the liver, and these lesions were considerably smaller than the lesions found in the wild-type mice.

The present invention therefore provides an animal model which is significantly more resistant to MHV, as well as to bacterial infections (e.g. salmonella).

Manipulation of the Ceacam1^{a} gene in mouse ES cells yielded one line of mice in which the expression of CEACAM1^{a} with 4 Ig domains is markedly downregulated. Comparatively, 2 Ig domain-expressing CEACAM1^{a} isoforms were increased 2 fold. Following intranasal inoculation of MHV-A59, homozygous mice that express low levels of the CEACAM1^{a} virus receptor develop fewer and smaller lesions in the liver than those found in the parental mice. The titers of virus produced in homozygous mice are decreased 50 fold and the virus is cleared from the liver within 7 days.

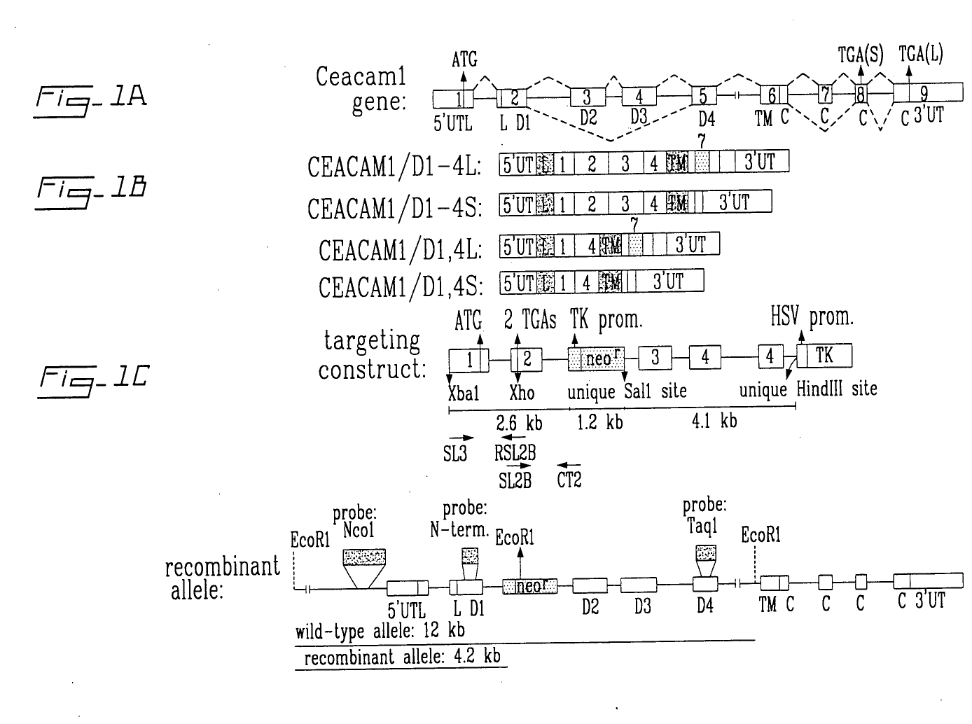

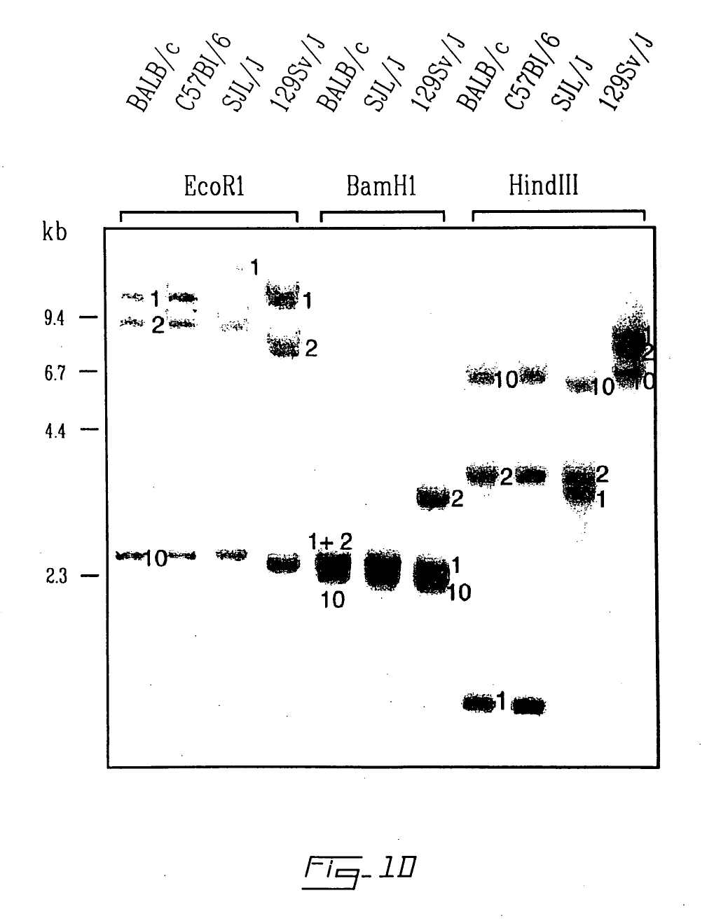

Using another strategy, CEACAM1^{a}−/− mice was also obtained. After two years of existence, the CEACAM1^{a}-targeted mice appear normal but appear to be fully resistant to pathogens gaining entry into cells through CEACAM1. Indeed, Ceacam1^{a}-targeted mice exhibiting a complete ablation of the CEACAM1^{a} protein were also generated. These mice will be referred to as −/− mice. Briefly, a different targeting construct was generated using the same source of 129Sv genomic DNA as described above in Example 3 and see below. The Ceacam1 targeting construct that gave rise to +/− ES cells is indicated in FIG. 1. Two independent +/− ES cell clones were microinjected into C57BI/6 mice to produce chimeric mice. Four male mice derived from each independent +/− ES cell line were mated with C57BI/6 females; one chimeric male mice produced from each ES cell line successfully contributed to the germline of offsprings. Heterozygous mice were mated and produced homozygous mice originating from each independent ES cell line. This mutation was also successfully transferred into the germline of 129Sv and BALB/c mice lineages.

The complete ablation of the CEACAM1^{a} protein was demonstrated using different methods which showed that the CEACAM1^{a} protein was completely absent from the tissues.

While some abnormalities were initially noted between −/− and +/+ mice, comparison with more animals (the noted abnormalities were based on the comparison of only two animals) showed that for −/−, mice maintained in pathogen-free conditions. There was no significant difference between −/− and +/+ mice with respect to neutrophils, lymphocytes, calcium levels in their serum, levels of sodium, potassium and chlorine ions in the urine or abnormalities in tissue sections of the colon, thymus and kidneys.

The present invention is illustrated in further detail by the following non-limiting examples.

### EXAMPLE 1

### Comparison of Ceacam Genes of Various Wild-Type Mice

Samples of 5 μg of genomic DNA extracted from different mouse strain is digested with either the EcoR1, BamH1 or HindIII restriction enzymes and run on 0.8% agarose gels. The DNA fragments were transferred to Gene Screen + and probed with a ³²P-labelled 395 bp Nco1 fragment located in the proximal promoter region. As illustrated in FIG. 1D, the three Ceacam-related genes Ceacam1 (1), Ceacam2 (2) and Ceacam10 (10) are identified. The numbers next to the various restriction fragments indicate the position of each gene in the particular digests of genomic DNA. Note that the 129Sv/J Ceacam2-specific EcoR1 restriction fragment is shorter than that of other mouse strains. The 1.7 kb Ceacam1-specific HindIII fragment present in BALB/c and C57BI/6 mice is not found in the 129Sv/J strain. Instead, this fragment migrates as an 8.0 kb fragment due to the mutation of the HindIII site present in intron 1 of other inbred strains.

It is apparent that the migration of restriction fragments corresponding to each gene varies slightly in different inbred mouse strains.

### EXAMPLE 2

### Identification and Characterization of the 129Sv Ceacam1^{a} Gene

The mouse 129Sv Ceacam1^{a} gene was isolated from a 129Sv genomic library, graciously provided by Drs. Christian Benoit and Diane Mathis (Strasbourg, France). This genomic library was prepared from the D3 ES cell line; partial Mbol restriction fragments of approximately 10-15 kb were inserted into a lambdaGEM12 vector. A total of 5×10⁵ pfu of this genomic library were screened with three distinct probes. A proximal promoter fragment (Nco1 probe; 395 bp fragment found at nucleotides 975-1370 in the Ceacam1^{a} promoter (24)) hybridizes to the murine Ceacam1^{a}, Ceacam1^{b}, Ceacam2 and Ceacam10 genes at high stringency. The Ceacam1^{a} gene was specifically detected using the full length Ceacam1^{a} cDNA or a Taq1 probe (nucleotides 1150-1295 in the Ceacam1^{a} cDNA) when hybridized at high stringency (24). Two clones encompassing the Ceacam1^{a} gene were obtained; the identity of the gene was confirmed by restriction digests and DNA sequence analyses of relevant exons.

As is illustrated in FIG. 1A, in the Ceacam1^{a} gene, the ATG initiation codon is located in the first exon, whereas two stop codons can be alternatively used, one in exon 8 (TGA(S): used in the translation of isoforms encoding a short cytoplasmic domain) and one in exon 9 (TGA(L): used in the translation of isoforms encoding a long cytoplasmic domain). The dashed lines over and under the gene structure represent the alternative splicing events producing CEACAM1^{a} isoforms with either two or four Ig domains and either a short or long cytoplasmic domain. Four nucleotide differences within the 5′ untranslated region located in exon 1 and two point mutations in the 3′ untranslated region in exon 9 were noted relative to the published sequence of the BALB/c Ceacam1^{a} cDNA (GenBank accession number X15351). No mutations were found in the coding region of the gene. However, a HindIII polymorphism between the BALB/c and 129Sv/J strains (FIG. 1D) was demonstrated by sequencing intron 1. Absence in the 129Sv/J mice of the HindIII site present in the first intron of the Ceacam1^{a} gene from BALB/c mice was demonstrated by an approximate 8.0 kb HindIII fragment in the 129Sv/J mice (FIG. 1D). The exon-intron distribution and the lengths of the introns were identical between the Ceacam1^{a} genes of the two mouse strains. Thus, this suggests that the neo cassette in intron 2 could be used to disrupt other strains of mice.

The four major alternatively splice variants produced by Ceacam1^{a} are illustrated in FIG. 1B. The CEACAM1^{a}/D1-4 variants encode the D1 to D4 Ig-like domains (identified in the boxes) that are linked through a transmembrane domain (TM) to either a short (S) or a long (L) tail. This alternatively spliced event is due to the inclusion of the 53 bp exon 7 (shaded, exon number over the box) resulting in a shift of the open reading frame and the translation of a 73 aa long tail. All Ceacam1^{a} splice isoforms express the N-terminal domain (D1) and most functions for this protein are mediated by this domain (5), except those of inhibitor of tumor development, intracellular signaling events and cytoskeletal associations.

### EXAMPLE 3

### Generation of Targeting Vector

The scheme for producing the targeting vector illustrated in FIG. 1C was designed to insert the pTK-neo^{r} selection cassette (31) downstream of exon 2. Two major restriction fragments were used to prepare the targeting vector. The first consisted of a 2.6 kb Xba1-Sal1 fragment encompassing the first two exons and the first intron of the mouse Ceacam1^{a} gene. This fragment served as a template for polymerase chain reactions to amplify the 5′ arm of the targeting vector consisting of two shorter fragments of 1.1 and 1.5 kb, respectively. Mutated nucleotides are indicated in bold in the following sequences. The mutated 1.1 kb fragment was produced by PCR amplifications with oligo SL3, located at the 5′ end of the 5′ untranslated region sense:5′CGGAGTATGTTCTAGAACACTG; SEQ. ID NO:1 and oligo RSL2B, located within exon 2 at the end of DNA sequence that encodes the signal sequence antisense: 5′GACTCGAGCAGTGGT-GGCAGGTCATCAGGAG; SEQ. ID. NO:2. The mutated 1.5 kb fragment was generated by PCR with oligo SL2B sense: 5′CTCCTGATGACCTGCCACCACTGCTCGAGTC; SEQ. ID. NO:3 and oligo CT2, located at a unique Sal1 site within intron 2 (antisense: 5′AGACACAATCTGTCGACTCCTC; SEQ. ID. NO:4). The SL2B and RSL2B oligos introduced 6 nucleotides as substitutions that encode two TGA stop codons in the open reading frame (bold in the oligo sequence) and a unique Xho1 site at the beginning of exon 2 which encodes the N-terminal domain, D1. The two fragments were joined together by overlapping PCR using oligos SL3 and CT2. The 2.6 kb 5′ arm of the targeting vector was linked to the pTK-neo^{r} selection cassette, originating from the pMC1-neo^{r}-polyA vector (31). The resulting 3.8-kb fragment was excised as a Not1 fragment and cloned upstream of the remaining 4.1-kb Ceacam1^{a} gene fragment that constituted the 3′ arm of the targeting vector. Finally, an HSV-tk negative selection cassette (31) was introduced downstream from the targeting vector in a unique HindIII site.

The verification as to whether the Ceacam1^{a}-targeted ES cell line DNA enclosed the TGA stop codons, PCR amplifications were performed on ES genomic DNA. An EcoR1-tagged oligo in the Ceacam1^{a} intron 1, located upstream of the stop codons (CT3: sense 5′GGAATTCCTAAGATTGATAGGTCTTTC; SEQ. ID NO:5) and another oligo positioned within the neo^{r} gene (RTKneo1: antisense 5′GGAAACATTCCAGGCCT; SEQ. ID NO: 6) were used in this procedure. The amplified 1.7 kb fragment was cloned and subjected to DNA sequence analyses. As is illustrated in FIG. 1C, the targeting vector had the TK-neo^{r} selection cassette inserted into a unique Sal1 site in the second intron.

Two other targeting vectors designed to eliminate the Ceacam1^{a} exon 2 were prepared. They principally differed in the position of the TK-neo^{r} selection cassette. In the first targeting construct tried, most of exon 2 was replaced with the 1.15 kb TK-neo^{r} selection cassette. In the other targeting vector, the 1.15 kb TK-neo^{r} selection cassette was inserted into the characteristic BamH1 site present further downstream in exon 2 (data not shown).

### EXAMPLE 4

### DNA Analyses

Genomic DNA was prepared from ES cell clones grown in 24-well dishes using standard procedures (31). Genotyping was performed using <1 cm of tails clipped from 3 week old pups; genomic DNA was prepared using a QiaAMP™ DNA minikit (Qiagen). Approximately 5 μg of genomic DNA was cleaved with the EcoR1 restriction endonuclease and separated on 0.75% agarose gels. The DNA was transferred to GeneScreen Plus™membranes (NEN-Life Science Products, Boston, Mass.) and hybridized at 42° C. for 18 h with 2-4×10⁶ dpm of random-primed alpha-³²P-dATP-labelled restriction fragments (24). Membranes were washed to a final stringency of 65° C. in a 0.1×SSC and 0.1% SDS solution. The Nco1 promoter fragment of 395 bp was used (24) to identify all three types of Ceacam genes. The full-length Ceacam1^{a} cDNA (20) was used for genomic library screening. N-terminal domain fragments specific either for the Ceacam1^{a} gene (102 bp) or a Ceacam2-specific fragment (153 bp), amplified by PCR as previously described (23,24), were used to distinguish the Ceacam1^{a} and the Ceacam2 genes. The 125 bp Taq1 restriction fragment located within exon 5 (FIG. 1C) is specific for the Ceacam1^{a} gene and does not hybridize with the Ceacam2 gene at high stringency. A 700 bp HindIII-Nael fragment prepared from the coding region of the neo^{r} gene was used to define the number of neo integration sites in the ES cell clones by Southern hybridization. A 360 bp cDNA fragment, corresponding to exons that encode the long or short cytoplasmic tails was also used to validate the integrity of the Ceacam1^{a} gene locus at its 3′ end.

### EXAMPLE 5

### ES Cell Culture

Twenty-five μg of the Not1-linearized targeting vector described in Example 3 were electroporated into 1×10⁷ of 129Sv R1 ES cells, graciously provided by Dr. Andras Nagy (Samuel Lunenfeld Research Institute, Toronto, Canada). ES cells were maintained on mitomycin-inactivated G418-resistant mouse embryonic fibroblasts in Leukemia Inhibitory Factor-containing D-MEM medium (Life Technologies, Grand Island, N.Y.) with 15% FBS (HyClone Laboratories, Logan, Utah) as previously described (31). G418 (300 μg/ml, active form) and Gancyclovir (2 μM) selections were applied 48 h post transfections, and clones were isolated and amplified after 9 days in selective medium.

### EXAMPLE 6

### Identification and Characterization of Positive Clones

Owing to the partial knockout phenotype and to avoid confusion between animals that may be generated with a complete Ceacam1^{a} gene ablation, the terminology +/+ for wild type mice, +/p for heterozygotes and p/p for the homozygous Ceacam1^{a}Δ4D mice described here will be used herein. One Ceacam1+/p ES cell clone (F3), using the targeting construct described in FIG. 1C, was identified from the 800 G418-resistant clones screened. No Ceacam1^{a} +/p ES cell line was obtained with the two other constructs out of the 3000 clones screened.

Analyses of EcoR1-cleaved genomic DNA the F3 ES cell line according to the procedure described in Example 4 revealed the characteristic Ceacam1^{a} targeted allele as illustrated in FIG. 1E. In a, the Nco1 probe hybridizes to all three mouse Ceacam-related genes (1, 2, 6). The Ceacam1^{a}-targeted allele is shortened from the original 12 kb (Ceacam1^{a} 129/sv) to 4.2 kb. In b, the neo probe detects one band of approx. 8.0 kb. Only one integration site was detected in the F3 ES cell line. The genomic DNA from +/+ mice did not hybridize with this probe, whereas the +/p and p/p mice were positive. In c, the Ceacam1-specific probe, located in the N-terminal domain of the gene (FIG. 1C), detected a 12 kb EcoR1 fragment in genomic DNA prepared from all mice except the p/p mice. In addition, this probe hybridized to the targeted 4.2 kb fragment in the F3 ES cell line and in the heterozygote and homozygote mice. In d, the Ceacam2-specific probe, also from the N-terminal domain of its respective gene, hybridized to either a 9.6 kb fragment or a 7.7 kb fragment depending on the contribution from the BALB/c or 129 Sv/J mice, respectively. The targeting event was also confirmed with other characteristic restriction digests of the F3 genomic DNA (data not shown).

The integrity of the mutant versus normal Ceacam1^{a} genomic locus was confirmed by hybridizing restriction fragments of ES genomic DNA with representative probes located 5′ or 3′ to the recombination site (data not shown). No modifications were found in the Ceacam2 or Ceacam10 genes in the ES cell line (FIG. 1E, panels a and d).

As the position of the selection cassette was located far from the engineered TGA stop codons, it was determined whether the targeted allele included the TGA stop codons using the PCR-based sequencing described in Example 3. It was determined that the recombination event had occurred between the stop codons and the selection cassette, thereby eliminating the stop codons from the F3 ES Ceacam1^{a}-targeted allele (FIG. 1C recombinant allele). Because the TGA stop codons were removed, there was a high probability that some CEACAM1^{a} proteins might be expressed from this targeted gene in homozygous mice.

### EXAMPLE 7

### Generation and Characterization of Chimeric Mice

Chimeric mice were generated by microinjection of the Ceacam1^{a}-targeted ES cells (at passage 20) into BALB/c blastocysts as described. Six chimeric male mice were obtained, only one of which transmitted the Ceacam1^{a} +/p targeted allele through the germline. The heterozygous Ceacam1^{a} +/p progeny mice were mated with either BALB/c or C57BI/6 females to produce homozygous Ceacam1^{a}(p/p) mice. Mating of heterozygous mice produced expected Mendelian ratios of Ceacam1^{a}p/p offspring (1.0+/+: 1.6+/p: 1.0 p/p). CEACAM1 is expressed in ovary and prostate; however, the progenies had approximately equal numbers of males and females (46.3% males: 53.7% females). Frequency of germline transmission was calculated to be 3.7% on the BALB/c background.

Experiments were performed on +/p and/or p/p Ceacam1^{a}-targeted mice and wild-type littermates with the same BALB/c genetic background.

Mice were genotyped according to the procedure described in Example 4. The non-targeted Ceacam1^{a} restriction fragment of 12 kb in the +/p mice appeared as half the hybridization intensity of the wild-type +/+ mice. This fragment was absent from genomic DNA of the p/p mice with a concomitant increase in the 4.2 kb targeted restriction fragment (FIG. 1E, panels a, b and c). Interestingly, in the Ceacam1^{a} p/p mice, the BALB/c Ceacam2 alleles were replaced by the 129Sv Ceacam2 alleles (FIG. 1E, panel d).

### EXAMPLE 8

### General Health Status of the Offsprings

A sizeable colony (approx. 350 individuals) of targeted mice was maintained for 18 months, on both the BALB/c and C57BI/6 backgrounds. No reduction of fertility, sex distortions, bone or cartilage abnormalities or abnormal behavior were noticed.

### EXAMPLE 9

### Sampling and Preparation of Tissues

The mice were sacrificed by cervical dislocation and the tissues were removed and washed in PBS. The intestine was dissected, cleared of debris and washed in PBS; it was then divided into sections of equal length corresponding to the duodenum, the jejunum and the ileum. The colon was sampled distal to the cecum. All tissues were immediately snap-frozen on dry ice for subsequent DNA, RNA or protein analyses or fixed in 4% paraformaldehyde/PBS and processed for histological analysis and immunohistochemistry.

### EXAMPLE 10

### Immunoblotting, Immunoprecipitations and Antibodies

Fresh tissues were excised from 2-6 month old Ceacam1+/+, +/p, or p/p mice, snap-frozen on dry ice and powdered using a mortar and pestle. The powder was resuspended in 500-1000 μl of lysis buffer. Proteins were separated on 8% SDS-PAGE gels and transferred to Immobilon membranes (Millipore, Nepean, ON). Expression of the CEACAM1 isoforms was detected by immunoblotting 75-200 μg of total proteins with the anti-CEACAM1-specific rabbit polyclonal Abs 231 or 2456 and ¹²⁵I-labelled protein A. To detect the CEACAM1 proteins expressing the long cytoplasmic domain, proteins were immunoprecipitated using 5 μg of a rabbit polyclonal Ab 836 IgG fraction. Immune complexes were collected, separated on SDS-PAGE gels and visualized using either ¹²⁵I-labelled protein A or ECL detection (Amersham Pharmacia Biotech, Baie d'Urfé, QC). Controls for the various splice isoforms were prepared from previously described NIH 3T3 cell clones that express Ceacam1^{a} cDNAs encoding four Ig domains with either the long or short cytoplasmic tail or alternatively constructs expressing two Ig domains CEACAM1^{a} proteins. Quantification of labeled proteins was done using a Fuji Biolmager 2000 system.

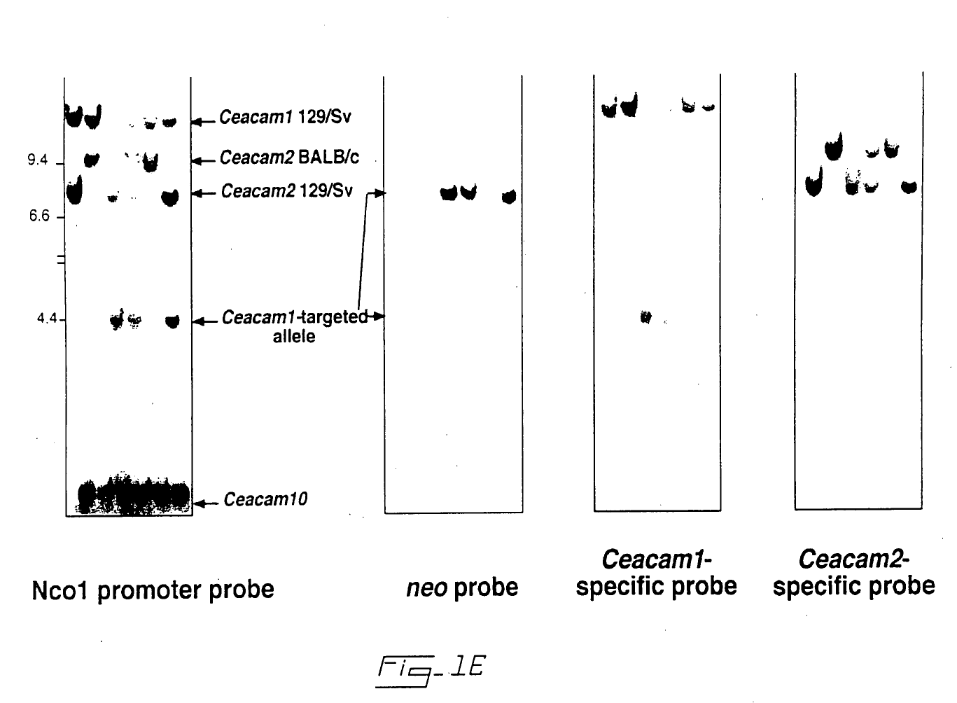

The expression of the CEACAM1^{a} isoforms in colon, liver and kidney tissues was thus examined from several different mice. A representative experiment is shown in FIG. 2. In these tissues, expression of the D1-4 isoforms was decreased in the Ceacam1^{a} p/p mice to 6±2%, 3±1% and 2±1% in colon, liver and kidney respectively, relative to CEACAM1^{a} expression in the wild-type +/+ control mice (FIGS. 2A and C). The expression of the D1,4 CEACAM1^{a} isoforms was also increased in these tissues, the highest being in colon with a 2.0 fold higher ratio. Interestingly, +/+ kidney tissue expressed a high D1-4>D1,4 CEACAM1^{a} ratio. Therefore, this tissue exhibited the most significant overall decrease of CEACAM1^{a} expression. These results suggest that insertion of the neo selection cassette in intron 2 of the Ceacam1^{a} gene modified the normal alternative splicing pattern, shifting it preferentially to expression of D1,4 Ceacam1^{a} mRNAs (see FIGS. 1A and B)

The expression of CEACAM1^{a}/D1-4-L isoforms was then determined (FIG. 2B). CEACAM1^{a}/D1-4-L expression was decreased in the p/p liver (FIGS. 2B and C; 9±4% of wild-type expression) and colon (data not shown; 15±4% of wild-type expression). A lack of discrimination of the splicing machinery relative to the expression of the cytoplasmic domains is apparent, as CEACAM1^{a}/D1-4-L is decreased in similar ratios as the CEACAM1^{a}/D1-4-S variants. These results also implied that Ceacam1^{a}-specific alternative splicing patterns differed slightly from tissue to tissue.

### EXAMPLE 11

### Histological Analyses

Tissues were excised from the mice, washed in PBS, fixed in 4% paraformaldehyde and processed for paraffin-embedding. Sections of 6 μm were prepared and subjected to immunohistochemistry with the anti-CEACAM1 polyclonal (Ab 231 or 2456) and monoclonal (MAb CC1) antibodies and counterstained with hematoxylin according to standard histological procedures. No apparent morphological differences were noted between colon, liver and kidney +/+ and p/p mice.

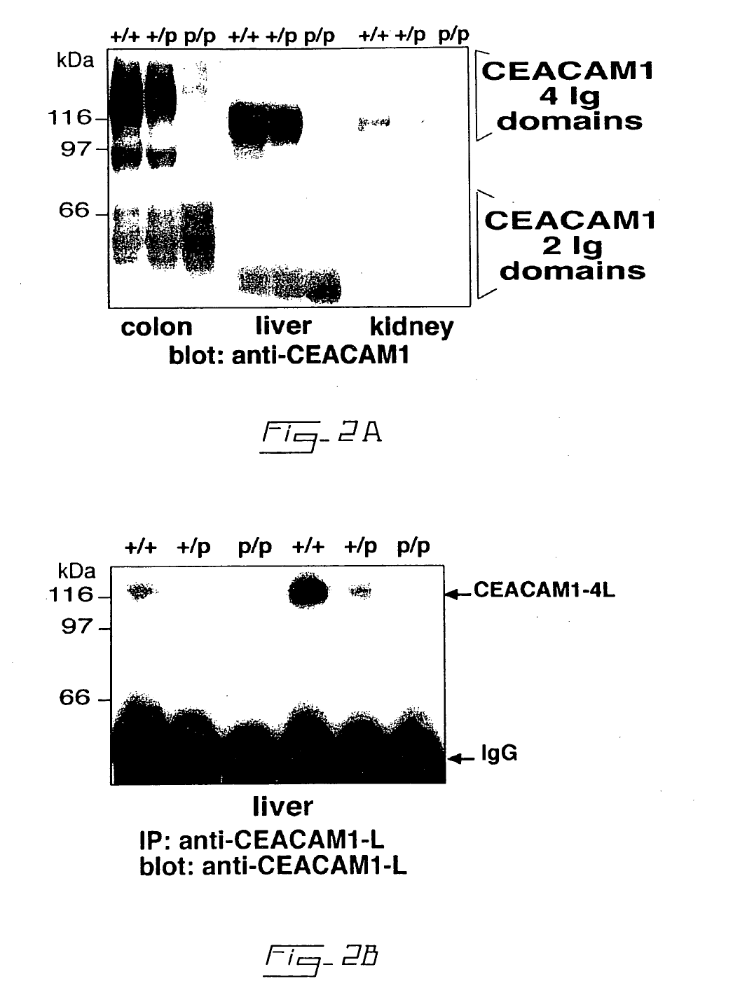

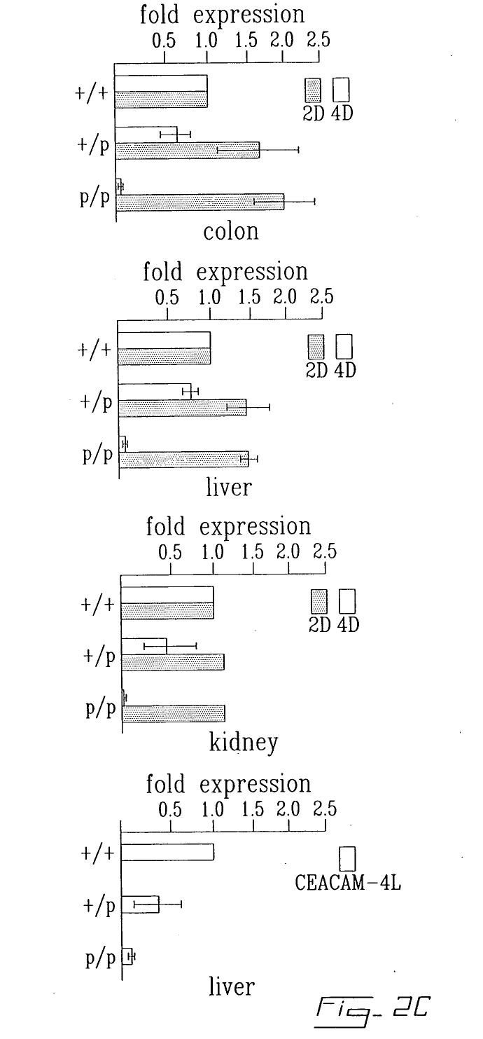

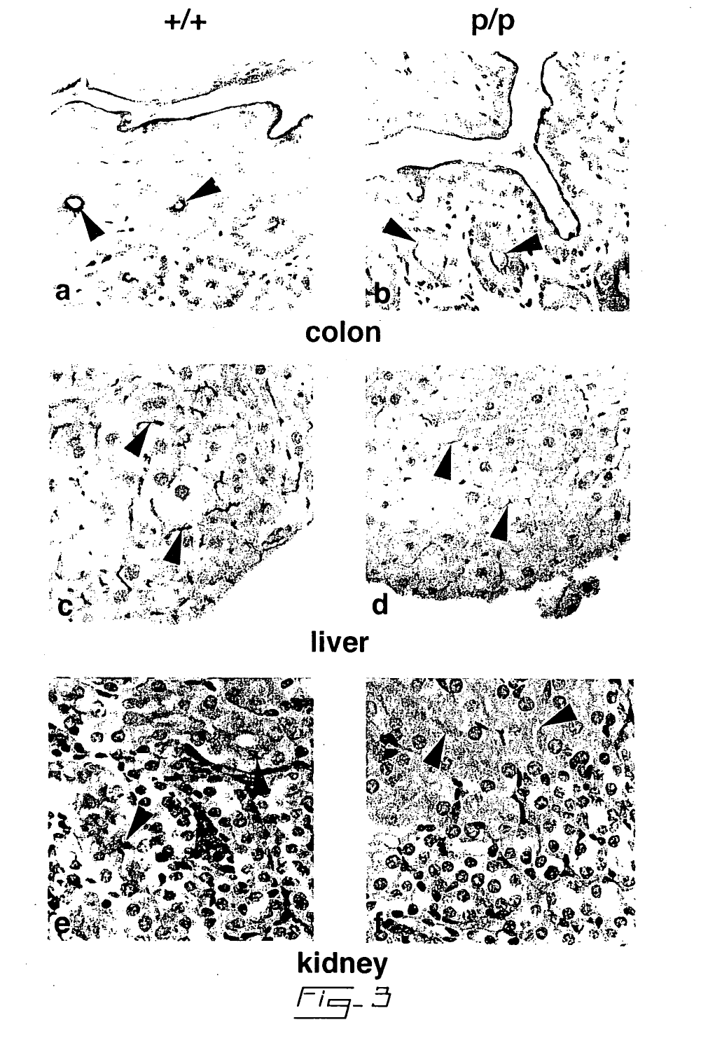

Panels a, c and e of FIG. 3 represent sections from the wild type +/+ mice, whereas Panels b, d and f are from the Ceacam1^{a}Δ4D p/p mice. In panels a and b presenting colon cross-sections, arrowheads point to the strong luminal in the +/+ mice and highlight the predominant staining at the luminal aspect of the +/+ colonic crypts that is fainter in the p/p mice but nevertheless present due probably to the expression of the 2 Ig domain-containing-isoforms (FIG. 3b). Antibody dilution experiments indicated that a 20-fold dilution of the anti-CEACAM1 antibody completely abrogated detection of the CEACAM1^{a} protein in the p/p colon, whereas positive staining was still observed in the +/+ mice with the same dilution.

In panels c and d, presenting liver cross-sections, hepatocyte bile canaliculi contacts (arrowheads) are CEACAM1-positive in the +/+ mice, whereas they only express CEACAM1 weakly or not at all in the p/p mice.

In panels e and f, presenting kidney cross-sections, collecting tubules of the kidney were strongly positive for CEACAM1^{a} in the +/+ mice (arrowheads), whereas those of the CEACAM1^{a}ΔaD mice were faint and generally negative. The magnification used for the sections is 40×.

Other tissues normally expressing CEACAM1^{a}(small intestine, endometrium, ovary, prostate, stomach, spleen, thymus and lung) also displayed weaker or no reactivity in the p/p mice. Sections of intestines from either the Ceacam1^{a}+/+, +/p or p/p mice were carefully examined in order to detect any possible intestinal structural alterations or differences in CEACAM1^{a} expression. The degree of leukocyte infiltration in lamina propria of either small or large intestine between p/p and wild-type mice was not very different. The epithelial layer appeared intact. There were normal numbers of goblet cells in the colon crypts. No loss of mucous from the goblet cells was observed. Similarly, no morphological changes were noticed in liver or kidney tissues excised from either younger (9 weeks-3 months) or older (5 months-1.5 year) p/p mice (data not shown).

### EXAMPLE 12

### Biochemical Analyses

CEACAM1^{a} is expressed in many tissues important for biochemical homeostasis such as liver, kidney and in the gastrointestinal tract. Because the decrease in expression of major CEACAM1^{a} isoforms might cause physiological imbalance, biochemical markers in serum samples taken from either fasting or non-fasting animals were analyzed. Urinalysis was also performed. Mice were anesthetized with a ketamine-xylazine-acepromazine mixture, and blood was collected from the jugular vein. Samples were processed for standard biochemical parameters at the Jewish General Hospital, Montreal, QC on a Hitachi Clinical Biochemistry Analyzer (model 917). No significant changes were noticed in characteristic enzyme markers, electrolytes, glucose, cholesterol or protein levels.

### EXAMPLE 13

### Hematological Analyses

CEACAM1^{a} is expressed in a number of blood cells (platelets, macrophages, granulocytes, B lymphocytes and activated T lymphocytes) (10,15). Because elimination or reduction of major CEACAM1^{a} isoforms might cause immunological deficiencies, the mice were subjected to hematological analyses. Blood was collected in heparinized tubes. Blood cells were counted and analyzed on a Baker CBC, model 9000 apparatus (Animal Resources Centre, McGill University). Blood samples from ten different siblings of +/+, +/p and p/p were tested for percentages of mature blood cells as well as total numbers of different blood cell types. Parameters were similar when siblings were compared.

### EXAMPLE 14

### Determination of CEACAM1^{a}Δ4D Susceptibility to MHV Infections

As CEACAM1 is a receptor for MHV strains (12), it was desirable to assess the status of the Ceacam1^{a}-targeted mice relative to MHV infections. All mice were inoculated and observed under code. Three month-old p/p mice and their +/p and +/+ siblings (in the BALB/c genetic background) were subjected to intranasal inoculation with 10⁶ pfu of the hepatotropic MHV-A59 virus strain (1×10⁴-1×10⁶ pfu/mouse), propagated in spontaneously transformed BALB/c 3T3 cell line in D-MEM supplemented with 10% FBS and non-essential amino acids (12). Infectivity of MHV-A59 was determined by plaque assays. The mice were observed daily for development of clinical signs of illness such as lethargy, ruffled fur, dehydration and paresis. They were sacrificed 3-31 days post-inoculation (dpi) or immediately if showing significant distress. The histopathology of the livers, immunostaining with anti-viral antibody and production of infectious virus from the livers of +/+, +/p and p/p mice inoculated with MHV-A59 were compared. Tissues were removed and processed as in Example 11. The number of lesions characteristic of liver infections were quantified by counting the number of necrotic areas observed in different microscope fields.

Signs of illness were readily apparent in +/+ normal BALB/c mice within 2 days after intranasal inoculation with 106 PFU/mouse. The animals were hunched, shivering, lethargic and had ruffled fur. In marked contrast, p/p mice inoculated with even 108 PFU/mouse did not show signs of illness up to 7 days after virus inoculation. Heterozygous +/p mice showed signs of virus disease at about 5 days after inoculation with 106 PFU/mouse, but most +/p mice did not succumb to infection. The wild type +/+ mice inoculated with 106 PFU/mouse either died or had to be euthanized for ethical reasons by day 3 or 4 after virus inoculation. Serum harvested at 31 dpi from +/p and p/p mice inoculated with 106 or 108 PFU/mouse MHV showed high titers of antiviral antibody by ELISA.

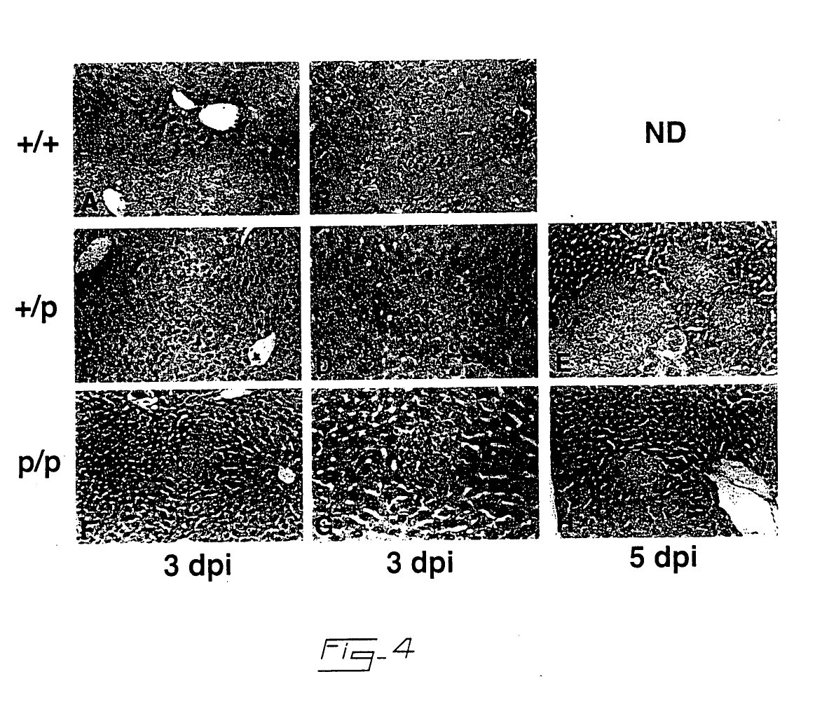

Lesions observed in the livers of these animals correlated with the severity of clinical signs (FIG. 4). Livers of +/+ mice 3 days after inoculation with 106 PFU/mouse had small patches of normal hepatocytes scattered among large confluent lesions. There was extensive coagulative necrosis with large areas of fibrin deposits, mild infiltration of mononuclear inflammatory cells and scattered apoptotic cells (FIG. 4, A and B). Livers of p/p mice 3 days after inoculation with 10⁶ PFU/mouse (FIG. 4, F and G) were mostly normal with scattered small focal lesions that consisted of small aggregates of inflammatory cells. Cells expressing viral antigens were found at the periphery of the lesions (data not shown). Few apoptotic cells were present and no large areas of necrosis were observed. Livers of +/p mice at 3 days after inoculation (FIGS. 4C and D) had lesions that were intermediate in severity between the +/+ and p/p livers. FIG. 4E shows that at 5 days after inoculation with 106 PFU/mice, liver lesions in +/p heterozygotes had increased in size and developed large areas of necrosis, while liver lesions in homozygous p/p mice remained small (FIG. 4H).

FIGS. 4A and B show very large areas of necrosis in liver from the wild type +/+ mice. C and D show intermediate-sized lesions in liver of heterozygous +/p mice; and F and G show scattered small aggregates of inflammatory cells (arrows) with no areas of hepatocyte necrosis. Heterozygous +/p mice (E) and homozygous p/p mice (H) were inoculated intranasally with 106 PFU of MHV-A59 virus and sacrificed at 5 dpi. At 5 dpi, areas of hepatocyte necrosis in +/p mice (F) had increased in size relative to the lesions at day 3 (C), while lesions in p/p liver remained very small. The magnification in these panels is as follows: A, C, E, F, H, 100×; B, D and G, 180×

Data on the number of lesions per section of liver are summarized in FIG. 5. Three experiments using an inoculum of 10⁶ PFU/mouse showed that +/+ mice had more lesions per unit area at 3 days after inoculation after which they died, while +/p heterozygotes survived and developed a larger number of lesions at day 5 after inoculation. Lesions were resolving or gone in +/p mice by day 7. In marked contrast, p/p mice had many fewer lesions than +/+ or +/p animals, most appearing by day 5, and being resolved by day 7. Interestingly, the number of lesions in livers of p/p mice at day 5 after inoculation did not increase even with a dose of 10⁸ PFU/mouse. When +/+ mice were inoculated with a low dose of 10⁴ PFU/mouse, they survived longer than +/+ mice given 10⁶ PFU/mouse, a lethal dose. At 10⁴ PFU/mouse and 5 days after inoculation, +/p mice had few signs of illness and markedly fewer lesions than +/p animals inoculated with 10⁶ PFU/mouse.

The small size and low numbers of lesions observed in livers of p/p mice at 5 days after inoculation with 10⁶ PFU/mouse correlated with the relatively low yield of infectious virus (0 to 2000 PFU/g) recovered from these livers. The livers of +/p mice 5 days after inoculation had much larger lesions than the p/p mice (FIG. 4) and had virus yields ranging from 850 to 120,000 PFU/g of liver, with an average of 22,100 PFU/g. Infectious virus was not recovered from livers of any of the surviving experimental animals on day 7 after inoculation, even in animals given 10⁸ PFU/mouse. Thus, altering the concentration and isoforms of MHV receptors expressed at the surface of hepatocytes in vivo greatly reduced the susceptibility of the hepatocytes to infection and ultimately reduced the severity of disease.

In FIG. 5, wild type +/+, heterozygous +/p and homozygous p/p mice were inoculated intranasally with 10⁴, 10⁶ or 10⁸ PFU of virus per mouse, and livers were harvested at 3, 5, or 7 days post inoculation (dpi). Several experiments are illustrated. The number of lesions per section of mouse liver is shown on the ordinate. Each bar indicates the number of lesions per liver section from one mouse that was harvested at the time indicated on the x-axis. Animals that had no liver lesions are indicated by the day of harvest on the x-axis. The +/+ animals inoculated with 10⁶ PFU died or were euthanized by day 3 or 4, whereas the +/p and p/p mice survived. The p/p mice were highly resistant to disease even at doses of 10⁸ PFU/mouse. ND indicates not done.

### EXAMPLE 15

### Alteration of the Level of CEACAM1^{a} Expression by Partial Knock-Put of the CEACAM1^{a} Gene

In the animal model reported above, the expression of the mouse Ceacam1^{a} gene in vivo was altered. It was shown that the expression of CEACAM1^{a}D1-4 isoforms with 4 Ig domains is markedly downregulated in a number of tissues in the targeted mice. Comparatively, expression of the CEACAM1^{a}D1,4 isoforms (with only domains 1 and 4) is increased 2 fold. In spite of the CEACAM1^{a} isoforms imbalance, the Ceacam1^{a}-targeted mice appear essentially normal since at least 24 months.

Three targeting vectors were used to abrogate the expression of the Ceacam1^{a} gene. These were based on the insertion of stop codons within exon 2 and replacement by the neo^{r} selection cassette of most or all of exon 2 (enclosing the D1 domain of the protein). It is unclear at present why this approach was problematic. Without being limited to a particular theory, one possibility is that the genomic DNA of the targeting construct was isolated from a library produced from 129 D3 ES cells. However three independent ES cell lines (J1, RW4 and R1) were transfected with these constructs without success. Since −/− ES cells having a complete knock-out of Ceacam1^{a} expression were also generated using a different targeting construct engineered with the same D3 genomic DNA (see Example 17), it appears that higher order genomic structures are likely to exist within intron 1 or exon 2 of the Ceacam1^{a} gene that prevent efficient recombination within this region.

Extensive characterization of the phenotypes of the homozygous p/p and heterozygous +/p Ceacam1^{a}Δ4D mice bred onto the backgrounds of two different inbred mouse lines has so far failed to reveal any significant structural or physiological differences between these and normal +/+ animals. Thus, although the total amounts of the two CEACAM1^{a}/D1-4 isoforms are decreased by about 90% in p/p animals, the residual CEACAM1^{a}/D1-4 proteins and/or the slightly increased levels of the two CEACAM1^{a}/D1,4 isoforms are probably able to provide sufficient amounts of CEACAM1 protein to accomplish the essential tasks of these glycoproteins during development.

Most experiments on the functions of CEACAM1 proteins have been carried-out using heterologous cell lines transfected with cDNAs encoding only a single CEACAM1 isoform. Yet, because CEACAM1 isoforms are expressed on most adherent cell lines, it is likely that the transfected cells used to express a recombinant CEACAM1^{a} glycoprotein are simultaneously expressing homologous CEACAM glycoproteins encoded by their own genome. Indeed, most mouse cell lines co-express more than one isoform of CEACAM1^{a}, and these may be found on the membranes as monomers, homodimers, or heterodimers. The sites on the cell membrane where each CEACAM1 isoform is expressed differ considerably from one cell type to another, and may vary with the physiological state of the cell. Most studies on functions of CEACAM1 proteins have focused either on the functions of the exodomains, or the functions of the cytoplasmic tails. There is as yet little information about the relative levels of expression and the functions of each of the 4 isoforms in different murine cell types and tissues.

Studies on cultured cell lines have shown that the level of expression of a virus receptor can affect the susceptibility of the cells to virus infection. In vivo, several experiments of nature suggest that reduced levels of expression of a virus receptor or co-receptor can also reduce susceptibility to virus infection and disease. For example, globoside is a receptor for human parvovirus. B19, and individuals who do not express this receptor are profoundly resistant to B19 infection (8). In addition, humans who are heterozygous for a mutant CCR-5 delta 32 allele that encodes a defective chemokine co-receptor for HIV-1 show increased resistance to virus infection and a decreased rate of disease progression (1). Experimentally, the biological significance of reduced levels of receptor expression as a determinant of virus susceptibility has been explored by using monoclonal antibodies to the receptor to block infection and also by constructing transgenic mice that express different levels of a human receptor for a specific virus (i.e. poliovirus) and comparing the specific infectivity of the virus for the different transgenic lines (27). The present invention is the first example of gene manipulation to explore the significance for virus susceptibility of reduced levels or altered ratios of splice variants of a virus receptor in the natural host of the virus.

Comparison of MHV-A59 infection of the Ceacam1^{a}Δ4D p/p, +/p and +/+ mice shows that the reduced expression and/or altered ratios of splice isoforms in the apparently healthy and phenotypically normal p/p mice has rendered the animals highly resistant to even very high doses (10⁸ PFU/mouse) of virulent, hepatotropic MHV-A59 delivered by a natural (intranasal) route of inoculation. The p/p animals develop fewer lesions than heterozygous +/p or wild type +/+ animals, have much smaller lesions with correspondingly less liver damage and lower titers of virus in the liver, and show little clinical evidence of virus infection. Perhaps transmission of MHV infection from a p/p mouse with a brief, inapparent infection to another p/p mouse would also be reduced due to the low titers of virus produced in p/p mice.

The mechanism(s) by which this manipulation of the Ceacam a gene makes the p/p mice resistant to MHV disease is not yet clear. Following an intranasal inoculation with virus, the virus first replicates locally in epithelial cells at the site of inoculation, then spreads to other tissues along nerves and/or through the blood stream either as free virions or in infected leukocytes (19). To reach the liver, virus from the blood would likely infect Kupffer cells and/or endothelial cells, and spread to hepatocytes causing expanding focal lesions. The diameter of the lesions in p/p mice was markedly smaller than that in +/+ mice or +/p heterozygotes, and lesions in livers of p/p mice expanded very little in diameter from day 3 to day 5 post inoculation while liver lesions in +/p mice expanded markedly from day 3 to day 5 (FIG. 4). The lower level and/or altered ratio of isoforms of CEACAM1^{a} in the p/p hepatocytes probably limits the spread of viral infection from hepatocyte to hepatocyte as well as the spread of virus to the liver. Another factor that may contribute to the larger sizes of the liver lesions observed in +/+ and +/p mice might be induction by MHV-A59 of monocyte procoagulant activity, a prothrombinase encoded by the fgl2 gene (21). Similar very large necrotic lesions in the liver are seen in MHV-3-induced fulminant hepatitis, and the pathogenesis of these lesions has been shown to be due to induction of fgl2 expression by MHV3 infection (11). Expression of fgl2 leads to microthrombus formation and hypoxia in the liver, rapidly followed by extensive necrosis and death. Perhaps because the yield of MHV-A59 in livers of infected p/p mice is much lower than the yield of virus in +/+ and +/p mice, the p/p mice may express less fgl2 than infected +/+ or +/p mice, and consequently have smaller liver lesions.

Presumably, the homozygous p/p animals are normal except for their high resistance to MHV infection (and other infectious agents). Transgenic plants that are resistant to multiple plant viruses have been engineered, based on transgenically-expressed viral proteins and/or post-transcriptional gene silencing. Genetically engineered, disease-resistant crops and animals can have substantial economic impact. MHV causes frequent epizootics in colonies of laboratory mice, and the virus can be persistently shed by immunosuppressed animals (9). Because inapparent MHV infection can alter the normal responses of mice to a variety of experimental procedures and cause death of some immunosuppressed animals, laboratory mouse colonies managers must invest in expensive surveillance programs to detect MHV infection. If an epizootic is detected, to eliminate the virus from infected colonies, breeding must cease and importation of new susceptible animals in a colony is prohibited for months. Valuable, irreplaceable mouse strains infected with MHV must be rederived by Ceasarian delivery, and sometimes all mice in an infected colony must be euthanized. Numerous strains of MHV that elicit strain-specific immune responses have been detected in mouse colonies, and animals are susceptible to repeated infections with different strains (9). All MHV strains appear to utilize CEACAM1^{a} isoforms as their principal receptors. Therefore, a strategy designed to reduce the availability of the receptor glycoproteins in inbred mice appears more likely to prevent epizootics of MHV than other approaches such as immunization. The present invention demonstrates the feasibility of genetically engineering inbred strains of mice for increased resistance to MHV, without introducing detectable changes in the development, physiology, fecundity or longevity of the inbred mice.

The MHV resistance experiments were confirmed using the C57BI/6 genetic background. Experiments using the 1295V background are underway. In view of the results with BALB/c and C57BI/6 of the p/p mice and with the C57BI/6−/− mice (see below), it seems clear that the susceptibility to MHV infection is not genetic background-specific.

### EXAMPLE 16

### Bacterial Infection of p/p Mice

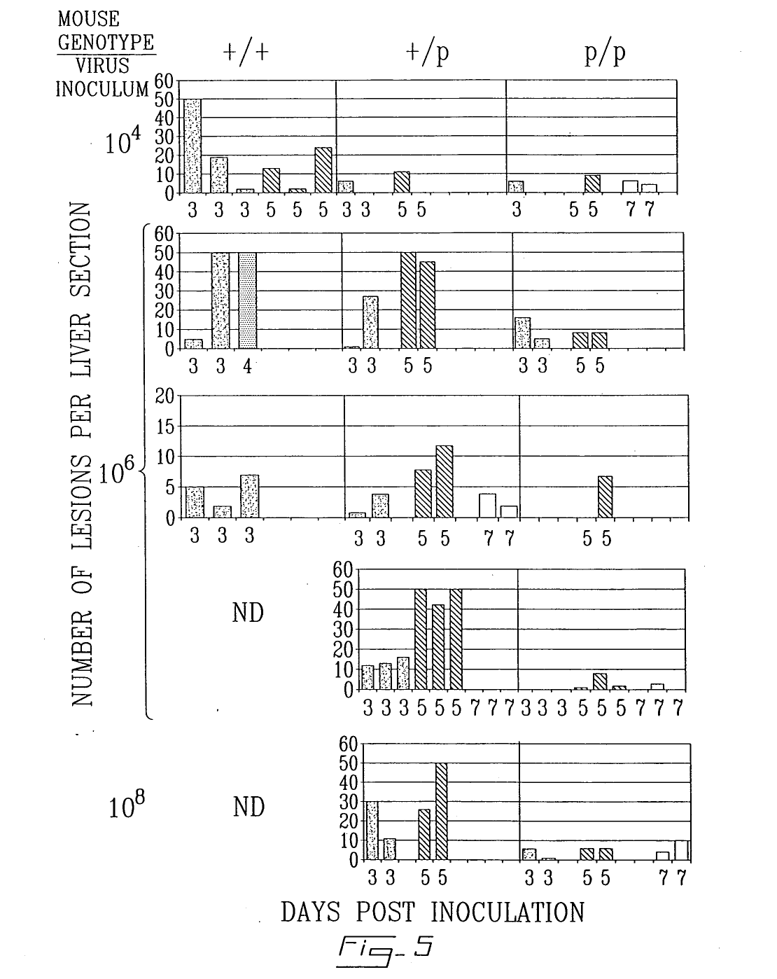

It was shown a number of years ago that the human CEACAM1 protein served as a receptor for type I fimbriae of bacteria such as Salmonella typhimurium and Escherichia coli (35). This interaction occurred with the human CEACAM1 D1 domain and was mediated by sugar moieties of the human CEACAM1 proteins. However, this interaction with these bacteria has never been investigated in the mouse. Since the human and mouse CEACAM1 proteins differ with respect to their glycosylation patterns, it could not be predicted whether the mouse CEACAM1 protein could also bind to these bacterial strains. A preliminary experiment was performed to investigate whether the p/p mice might behave differently than wild-type mice with respect to bacterial infection. A survival experiment was performed using the following procedure. A statistically significant number of +/+, +/p and p/p mice (12, 13 and 10 mice respectively, with equal distribution of males and females in each family) were intravenously injected with 10² PFU of infectious Salmonella enteritidis. Survival of these infected mice was observed over the next 13 days post-infection. As seen in FIG. 6, all mice showed decreased survival as of day 5 post-infection. However, only 8% of the +/+ mice and 15% of the +/p mice survived past this point, whereas 40% of the p/p mice were still alive 13 days post-infection. This experiment suggests that the mouse CEACAM1 protein also binds to Salmonella and that modification of the CEACAM1 isoform ratios in vivo in the p/p mice alters the ability of the bacteria to bind and infect the mice. Similar experiments using E. coli to verify the effect of the CEACAM1 genotypes on an additional type of bacterial infections have been carried-out. In any event, in view of the modulation of the resistance of mice to pathogens gaining entry to a cell via CEACAM1, the present invention validates the transgenic animals of the present invention as model systems for the resistance thereof to pathogens using CEACAM1, parts thereof or isoforms thereof as a receptor. For certainty, the transgenic animals (and tissues and cells thereof) also serve as model systems for CEACAM1 functions other than resistance to pathogens.

### EXAMPLE 17

### Generation of Targeting Vector for the Complete Ablation of Ceacam1^{a}

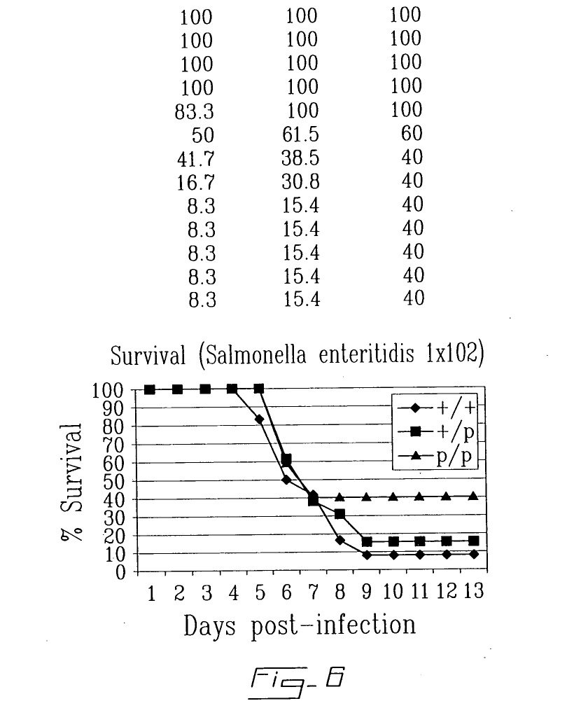

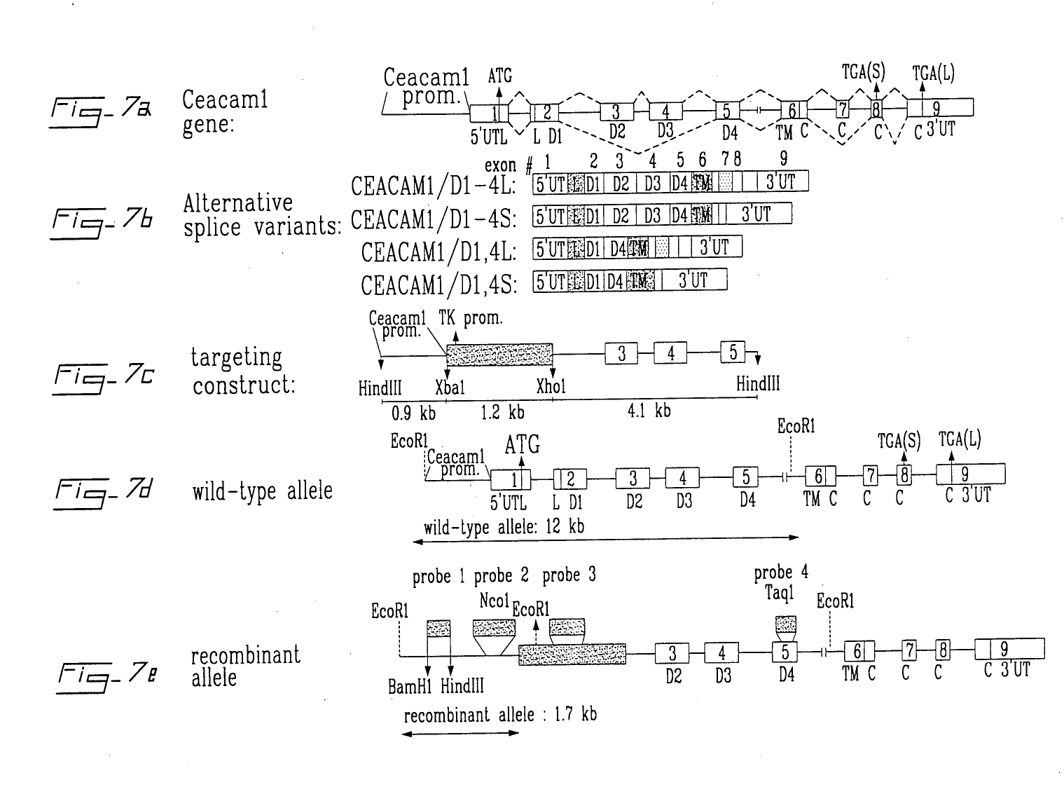

The scheme for producing the targeting vector illustrated in FIG. 7c was designed to replace exons 1 and 2, all of intron 1 and part of intron 2 of the 129Sv Ceacam1^{a} gene (FIG. 7a) by the insertion of the pTK-neo^{r} selection cassette (31). Two major restriction fragments were used to prepare the targeting vector. The first consisted of a 0.9 kb HindIII-XbaI fragment encompassing part of the promoter of the mouse Ceacam1^{a} gene. This fragment was cloned upstream of the pTK-neo^{r} selection cassette, originating from the pMC1-neo^{r-}polyA vector (31). The resulting 2.1-kb fragment was then cloned upstream of the remaining 4.1-kb Ceacam1^{a} gene fragment that constituted the 3′ arm of the targeting vector.

### EXAMPLE 18

### DNA Analyses

Genomic DNA was prepared from ES cell clones grown in 24-well dishes using standard procedures (31). Genotyping was performed using <1 cm of tails clipped from 3-week old pups; genomic DNA was prepared using a QIAamp DNA kit (Qiagen). Approximately 5 μg of genomic DNA was cleaved with the EcoR1 restriction endonuclease and separated on 0.75% agarose gels. The DNA was transferred to GeneScreen Plus membranes (NEN-Life Science Products, Boston, Mass.) and hybridized at 42° C. for 18 h with 2-4×10⁶ dpm of random-primed alpha-³²P-dATP-labelled restriction fragments (24). Membranes were washed at a final stringency of 65° C. in a 0.1×SSC and 0.1% SDS solution. The Nco1 promoter fragment of 395 bp (FIG. 7e, probe 2) was used as a probe (24). Positive results were confirmed using a 93 bp BamH1-HindIII fragment (FIG. 7e, probe 1) found within the Ceacam1^{a} promoter in a region located outside the targeting vector. A 700 bp HindIII-NaeI fragment (FIG. 7e, probe 3), prepared from the coding region of the neo^{r} gene, was used to define the number of neo integration sites in the ES cell clones by Southern hybridization. The 125 bp Taq1 restriction fragment located within exon 5 (FIG. 7e, probe 4), specific for the Ceacam1 gene, was used to confirm the integrity of the 3′ portion of the recombined gene.

### EXAMPLE 19

### ES Cell Culture and Generation of Chimeric Mice

Twenty-five pg of the Not1-linearized targeting vector described above were electroporated into 1×10⁷ of 129Sv R1 ES cells, graciously provided by Dr. Andras Nagy (Samuel Lunenfeld Research Institute, Toronto, Canada). ES cells were maintained on mitomycin-inactivated G418-resistant mouse embryonic G418-resistant fibroblasts in Leukemia Inhibitory Factor-containing D-MEM medium (Life Technologies, Grand Island, N.Y.) with 15% fetal bovine serum (FBS) (HyClone Laboratories, Logan, Utah) as previously described (31). G418 (300 μg/ml, active form) was applied 48 h post transfections, and clones were isolated and amplified after 7 or 8 days in selective medium.

### EXAMPLE 20

### Generation and Breeding of Chimeric Mice

Chimeric mice were generated by microinjection of the Ceacam1^{a}-targeted ES cells (at passage 20) into C57BI/6 blastocysts as described (12). Chimeric males were crossed with either the C57BI/6, BALB/c or 129Sv females. Heterozygous mice were obtained and crossed to generate Ceacam1^{a} homozygous mice. Experiments were performed on +/− and/or −/− Ceacam1^{a}-targeted mice produced on a C57BI/6 background and wild type (+/+) C57BI/6 mice of the same genetic background.

### EXAMPLE 21

### Sampling and Preparation of Tissues

The mice were sacrificed by cervical dislocation, and the tissues were removed and washed in PBS. The intestine was dissected, cleared of debris and washed in PBS; it was then divided into sections of equal length corresponding to the duodenum, jejunum and ileum. The colon was sampled distal to the cecum. All tissues were immediately snap-frozen on dry ice for subsequent DNA, RNA or protein analyses or fixed in 4% paraformaldehyde/PBS and processed for histological analysis and immunohistochemistry.

### EXAMPLE 22

### Histological Analyses

Paraformaldehyde-fixed tissues were dehydrated in ethanol and paraffin-embedded. Tissue sections of 6 μm thickness were stained with anti-CEACAM1 antibodies (19) and counterstained with hematoxylin according to standard histological procedures.

### EXAMPLE 23

### RNA Preparation and Northern Analysis

Tissues were retrieved from the mice and snap-frozen on dry ice. The tissues were then powdered using a mortar and pestle kept at −80EC and the RNA was extracted using materials provided in the RNAqueous kit (Ambion) following manufacturer's recommendation. 5 μg of total RNA was subjected to electrophoresis in formaldehyde-agarose gels and transferred to Hybond N+ (Amersham). The membrane was hybridized with a full-length ³²P_labelled Ceacam1^{a} cDNA for 18 hrs at 42EC and washed in a solution of 0.1×SSC+0.1% SDS at 65EC. Membranes were exposed to X-ray films for 18 hrs or 96 hrs.

### EXAMPLE 24

### Antibodies, Immunoblotting, and Immunoprecipitations

Fresh tissues were excised from 2-6 month old Ceacam1^{a}+/+, +/−, or −/− mice, snap-frozen on dry ice and powdered using a mortar and pestle. The powder was resuspended in 500-1000 μl of lysis buffer. Proteins were separated on 8% SDS-PAGE gels and transferred to Immobilon membranes (Millipore, Nepean, ON). Expression of the CEACAM1 isoforms was detected by immunoblotting 75-200 μg of total proteins with the anti-CEACAM1-specific rabbit polyclonal Abs 231 or 2456. The control used in the experiments was a cell lysate from CEACAM1^{a}-transfected NIH 3T3 cells. Immune complexes were visualized using an ECL detection system (Amersham Pharmacia Biotech, Baie d'Urfé, QC).

### EXAMPLE 25

### Biochemical and Hematological Analyses

Mice were anesthetized with a ketamine-xylazine-acepromazine mixture, and blood was collected from the jugular vein. Samples were processed for standard biochemical parameters on a Hitachi Clinical Biochemistry Analyzer (model 917). For hematological studies, blood was collected in heparinized tubes. Blood cells were counted and analyzed on an ADVIA 120 apparatus (CTBR Bioresearch, Pointe-Claire, Qc).

### EXAMPLE 26

### Mouse Hepatitis Virus Preparations and Intranasal Inoculation of Mice

The MHV-A59 virus strain used in these experiments was propagated in the spontaneously transformed 17 Cl 1 line of BALB/c 3T3 cells as previously described (26). The supernatant medium was collected at 24 hours after inoculation, centrifuged to remove cellular debris, aliquoted, quickly frozen and stored at −80EC. Titers of infectious virus were determined by plaque assay in 17 Cl 1 cells (26). Ceacam1^{a}−/−, +/− and +/+ mice 8 to 12 weeks old were inoculated intranasally with 15 μl of virus containing 10⁸ plaque forming units (PFU) in DMEM with 10% FBS. Control, uninfected +/+, +/− or −/− mice were sham-inoculated intranasally with 15 μl of DMEM with 10% FBS: The mice were observed daily for clinical signs of illness, such as lethargy, ruffled fur, hunched posture or paresis. A numerical scale of clinical symptoms was used to assess the degree of illness of the animals.

### EXAMPLE 27

### Analyses of Tissues of Inoculated Mice

At intervals of 2, 4, and 6 days after inoculation, mice were sacrificed, serum samples were collected for evaluation of anti-viral antibody and the livers and brains were collected and processed to determine the yield of infectious virus and to study histopathology. To quantitate the infectious virus in the liver, portions of the liver removed at necropsy were rinsed in Dulbecco and Vogt phosphate buffered saline, weighed, homogenized in D-MEM with 10% FBS, and rapidly frozen and thawed at 37° C. three times. Cell debris was removed by centrifugation. The virus titer per gram of liver in the supernatant medium was determined by plaque assay on 17 Cl 1 cells as described previously. Tissues were fixed in neutral buffered formalin or 4% paraformaldehyde, embedded in paraffin, sectioned and stained with hematoxylin. Sections were examined by light microscopy, and the number and sizes of lesions in comparable areas of the liver sections were determined. Virus-infected cells in the liver were identified by immunostaining with a monoclonal antibody directed against the viral nucleocapsid protein N, kindly provided by Julian Leibowitz, (Texas A&M University, College Station, Tex.), followed by peroxidase-labeled anti-mouse Ig. Controls, which showed no immunostaining, included antibody treatment and peroxidase-conjugated anti-mouse IgG treatment of liver sections from sham-inoculated mice, and incubation of liver sections from infected mice with a monoclonal antibody directed against an irrelevant antigen, followed by peroxidase-conjugated anti-mouse IgG.

### EXAMPLE 28

### Generation of Ceacam1^{a}-Targeted ES Stem Cells and Mice

The strategy leading to complete abrogation of expression of CEACAM1^{a} in mice was based on the removal of the first two exons of the Ceacam1a gene (FIGS. 7a and c), shown to encode the 5′ untranslated region of the gene as well as the leader sequence and the N-terminal domain. The initiator AUG codon is positioned in the first exon of this gene. For this purpose, a Xba1-Xho1 restriction fragment encoding these two exons was removed from the gene and the cassette encoding the TK (thymidine kinase) promoter and the neo^{r} gene was inserted in these same sites (FIG. 7c).

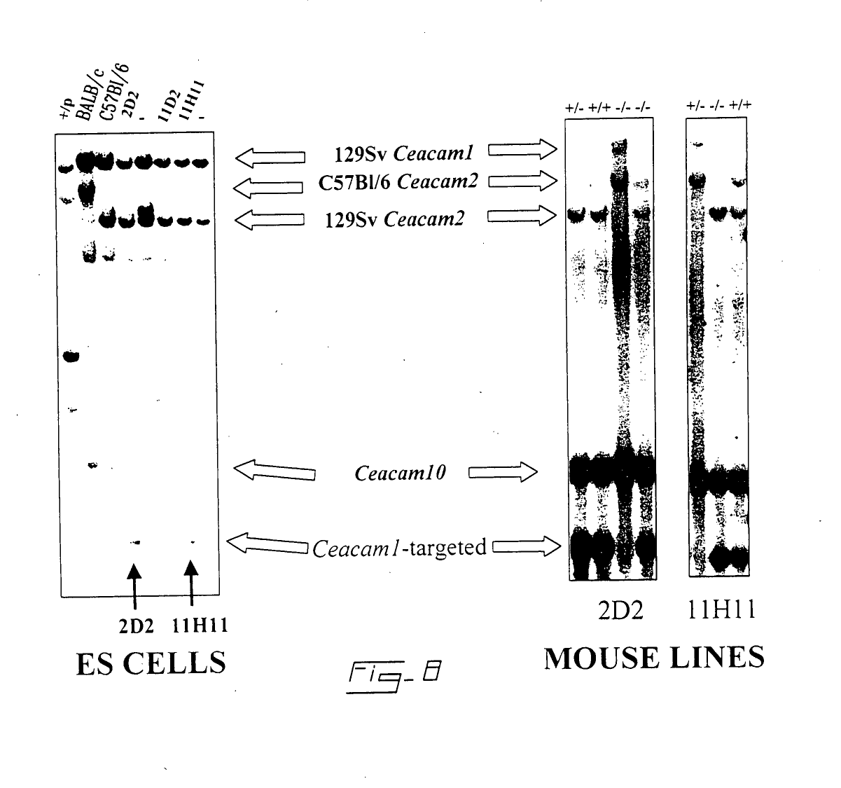

The targeting vector was electroporated into mouse R1 ES stem cells that grew on feeder layers of mitomicyn C-inactivated G-418-resistant fibroblasts in the presence of Geneticin as previously described (31). 830 clones were isolated after selection for 7 or 8 days in the G418-containing medium. DNA was prepared from these clones and the targeting event was evaluated by Southern analyses of EcoR1-digested genomic DNA using a ³²P-labelled BamH1-HindIII probe located in the Ceacam1^{a} promoter, in an region upstream of that encompassed in the targeting vector. In +/− ES stem cells, this would give rise to a recombinant 1.7 kb genomic band, due to the insertion of the neo^{r} gene carrying a novel EcoR1 site (FIGS. 7d and e). 33/830 clones were shown to have sustained the recombination event (FIG. 8). The Southern blot was also hybridized with a ³²P-labelled probe specific to the neo^{r} gene to confirm the targeting event and the number of integration sites in the +/− ES cell clones. (data not shown). The targeting event was also confirmed with other characteristic restriction digests of the 2D2 and 11H11 ES cell genomic DNA (data not shown). The integrity of the mutant versus normal Ceacam1^{a} genomic locus was confirmed by hybridizing restriction fragments of ES genomic DNA with representative probes located 5′ or 3′ to the recombination site (data not shown).

Chimeric mice were generated by microinjection of the ES cell lines (2D2 and 11H11) into C57BI/6 mouse blastocysts. Eight chimeric male mice were obtained, four of which transmitted the Ceacam1^{a}+/− targeted allele through the germline. The heterozygous Ceacam1^{a}+/− progeny mice were mated to produce homozygous (−/−) mice. Mice were genotyped by Southern analyses with the BamH1-HindIII promoter probe (FIG. 8). The non-targeted Ceacam1^{a} restriction fragment of 12 kb in the +/− mice appeared as half the hybridization intensity of the wildtype +/+ mice. This fragment was absent from genomic DNA of the −/− mice with a concomitant increase in the 1.7 kb targeted restriction fragment (FIG. 8). The frequency of germline transmission was calculated to be 22% on a mixed background (C57BI/6, BALB/c, 129Sv). Mating of heterozygous mice produced expected Mendelian ratios of Ceacam1^{a} −/− offspring (1.0+/+: 1.8+/−: 0.9−/−). CEACAM1 is expressed in ovary and prostate; however, the progenies had approximately equal numbers of males and females (53% males: 48% females).

A sizeable colony (approx. 350 individuals) of targeted mice were maintained for one year, on the BALB/c, 129Sv and C57BI/6 backgrounds and no reduction of fertility, bone or cartilage abnormalities, tumors or abnormal behavior were noticed.

### EXAMPLE 29

### Expression of CEACAM1 Glycoproteins in Ceacam1^{a}-Targeted Mice

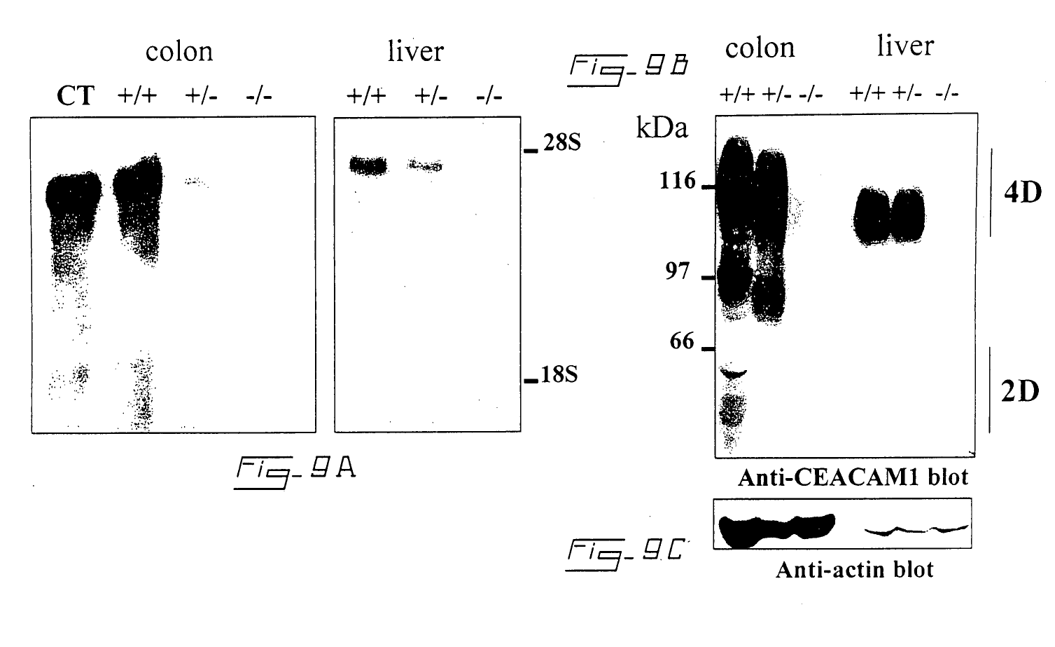

The complete abrogation of CEACAM1 expression was first verified at the transcriptional level. Total RNA was prepared from colon and liver of several mice from each litter and separated on formaldehyde-agarose gels. Northern blots were produced and subjected to hybridization with the ³²P-labelled full length Ceacam1^{a} cDNA. The wild-type and heterozygous mice produced a 4 kb RNA band corresponding to the Ceacam1a transcript. The intensity of RNA fragment was approximately half as that noticed with the control RNA produced from wild-type mice. No Ceacam1^{a} transcript was revealed in the homozygous mice, indicating that the gene inactivation strategy completely abrogated Ceacam1^{a} transcriptional activity (FIG. 9A). Correspondingly, we examined the expression of the CEACAM1a protein isoforms in mouse colon and liver tissues by immunoblotting total proteins from several different mice with monoclonal and polyclonal anti-CEACAM1a antibodies (FIG. 9B). In both these tissues and others (data not shown), expression of all CEACAM1a isoforms in the homozygous mice was completely eliminated relative to expression in the wild type +/+ or heterozygous mice (FIG. 9B). Actin protein levels were constant in these tissues (FIG. 9C).

### EXAMPLE 30

### Histological Data Comparisons Using the −/− Mice

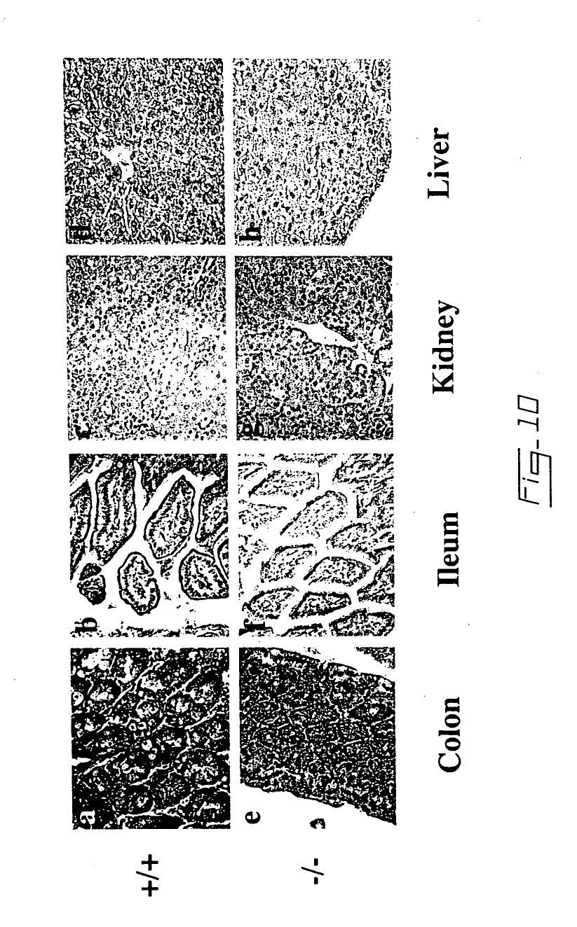

Paraffin-embedded tissue sections were stained for routine histology and immunostained with anti-CEACAM1 polyclonal (Ab 2456) and monoclonal (MAb-CC1) antibodies. No histological differences were noted in the colon, small intestine, liver, kidney, prostate, ovaries, uterus, brain, lungs, heart and spleen of −/− mice as compared to those of +/+ mice (FIG. 10). When immunostained with a polyclonal anti-CEACAM1^{a} antibody, tissues from wild type +/+ animals exhibited strong expression of the luminal membrane surface and crypt colonic and intestinal epithelium (FIGS. 10a and b, top row). Bile canaliculi of the liver and proximal tubules of the kidney were also strongly positive for CEACAM1^{a} in +/+ mice (FIGS. 10c and d, top row). In contrast, in −/− mice, colonic (FIG. 10e) or intestinal (FIG. 10f) epithelial cells revealed no staining of the crypts, in spite of long development of the immunostaining. Hepatocytes (FIG. 10h) or the collecting tubules in the kidney (FIG. 10g) of −/− mice were not labeled with anti-CEACAM1^{a} antibodies. Other tissues that normally express CEACAM1^{a}(small intestine, endometrium, ovary, prostate, stomach, spleen, thymus and lung) also displayed no immunostaining in the −/− mice. No histological abnormalities were observed in any of these tissues.

The −/− mice had no obvious morphological differences from +/+ mice, and were equally fertile. No increase in the incidence of tumors relative to +/+ mice was observed as −/− animals aged. Taken together, the studies described above show that the phenotypes of Ceacam1^{a}−/− mice are similar to those of normal wild type +/+ mice.

### EXAMPLE 31

### Biochemical and Hematological Analyses Comparisons Using the −/− Mice

CEACAM1^{a} is expressed in many tissues important for biochemical homeostasis such as liver, kidney and in the gastrointestinal tract. Because the abrogation in expression of major CEACAM1^{a} isoforms might cause physiological imbalance, biochemical markers in serum samples taken from non-fasting animals were analyzed. Urinalysis was also performed. No significant consistent changes were noticed in characteristic enzyme markers, electrolytes, glucose, cholesterol or protein levels.

CEACAM1^{a} is expressed in a number of blood cells (platelets, macrophages, granulocytes, B lymphocytes and activated T lymphocytes). Because elimination of major CEACAM1^{a} isoforms might cause immunological deficiencies, the mice were subjected to hematological analyses. Blood samples from four different litters (13 mice) of +/+, +/− and −/− were tested for percentages of mature blood cells as well as total numbers of different blood cell types. Parameters were similar when siblings were compared.

### EXAMPLE 32

### Mouse Hepatitis Virus Infections

In view of the results obtained with p/p mice, the complete abrogation of CEACAM1 expression should greatly influence the ability of the virus to infect the Ceacam1^{a}−/− mice. To verify this, +/+ and −/− mice of the 11H11 Ceacam1^{a}-targeted strain were inoculated intranasally with 108 PFU of MHV-A59 viral particles. With this amount of virus, the +/+ mice were moribond 2 days post-inoculation and were sacrificed. All parameters tested in the +/+ animals indicated a massive infection with MHV had taken place. Although they showed no signs of discomfort, the homozygous mice were also sacrificed, but at 4 or 6 days post-inoculation and the condition of their livers and brains were evaluated. No viral lesions were observed in any liver or brain section examined. Furthermore, no infectious viruses were recovered from the liver or brain tissues. Neither viral antigens nor viral RNA were detected in these tissues by immunohistochemistry or RT-PCR respectively. Viral titers were examined in lung extracts and no virus was found in this tissue either. As well, no viral RNA was present as evaluated by RT-PCR amplifications of lung tissue extracts.

### EXAMPLE 33

### Ablation of CEACAM1^{a} Expression in Mice

These animals are housed in a pathogen-free facility where they should not encounter bacterial or viral challenges. There is now gathering evidence in the CEACAM1 literature that, when the immune system of humans is challenged with Neisseria gonorrhea or other bacterial pathogens, activated T cells and dendritic cells will increase the expression of CEACAM1 at the cell surface where it serves as a negative regulator of cell surface receptors (36-40). Experiments are currently being carried out to verify whether the absence of CEACAM1^{a} from the mice might likely lead to hyperproliferation of either T and/or B lymphocytes as these cells will have lost one of the major negative regulator.

In any event, the results presented herein strongly indicate that the elimination of CEACAM1^{a}, the unique mouse hepatitis virus receptor, from the animals prevents any MHV infection from taking place: (1) no viral particles were detected; (2) or viral RNA or protein was revealed in the tissues that serve as targets for the MHV-A59 virus in spite of the overwhelming inoculum that leads to death of the +/+ mice in 2 days. Taken together, the data presented herein indicates that the Ceacam1^{a}−/− mice appear fully resistant to Mouse Hepatitis Virus infections.

Although the present invention has been examplified with mice strains 129Sv/J and BALB/c with CEACAM1^{a}, it should be clear that the invention covers all mice strains containing CEACAM1^{a} and furthermore that CEACAM1⁶ disruption should also be shown to lower the infectious potential of MHV and other pathogens which use CEACAM1.

Of note, when the p/p mice were inoculated intracranially with the MHV virus (another inoculation route), they developed demyelination. These injected mice exhibited negligible symptoms whereas the +/+ mice died or were paralyzed by the same dose. IC inoculations of the −/− mice are currently underway using a considerable amount of MHV virus (a concentration far exceeding that required to kill the control mice). It is expected that these intracranially injected −/− mice will also show negligible symptoms as the experiments performed with the p/p mice.

Although the present invention has been described hereinabove by way of preferred embodiments thereof, it can be modified without departing from the spirit and nature of the subject invention as defined in the appended claims.

### REFERENCES

1. Balfe et al., 1998, AIDS Res. Hum. Retroviruses 14:1229-134.

2. Barnett et al., 1989, J. Cell Biol. 108: 267-276.

3. Barthold et al., 1990, Lab. Anim. Sci. 40:133-137.

4. Barthold et al., 1993, Lab. Anim. Sci 43:276-284.

5. Beauchemin et al., 1998, Role of C-CAM as a tumor suppressor. p. 155-175. In Stanners, C. P. (ed.), Cell Adhesion and Communication Mediated by the CEA Family. Harwood Academic Publishers, Amsterdam.

6. Beauchemin et al., 1999, Exp. Cell Res. 252, 243-249.

7. Bradley, A., 1987, Production and analysis of chimeric mice. In Robertson, E. J. (ed.), Teratocarcinomas and Embryonic Stem Cells: A Practical Approach. IRL Press, Oxford.

8. Brown et al., 1994, N. Engl. J. Med. 330:1192-1196.

9. Compton et al., 1993, [published erratum appears in Lab Anim Sci 1993 43 (2):203]. Lab. Anim. Sci. 43:15-28.

10. Coutelier et al., 1994, Eur. J. Immunol. 24:1383-1390.

11. Ding et. al., 1997, [published erratum appears in J. Virol. 1998 April;72(4):3504]. J. Virol. 71:9223-9230.

12. Dveksler et al., 1991, J. Virol. 65:6881-6891.

13. Dveksler et al., 1993, J. Virol. 67:1-8.

14. Dveksler et al., 1993, Proc. Natl. Acad. Sci. USA 90:1716-1720.

15. Godfraind et al., 1995, 73:615-627.

16. Holmes et al., 1994, Coronavirus Receptors, S. Siddell (ed.), The Coronaviruses. Springer-Verlag, Vienna.

17. Homberger et al., 1992, Lab. Anim. Sci. 42:347-351.

18. Hsieh et al., 1995, Cancer Res. 55: 190-197.

19. Lavi et al., 1988, Lab. Invest. 58:31-36.

20. McCuaig et al., 1993, Gene, 127, 173-183.

21. McGilvray et al., 1998, J. Biol. Chem. 273:32222-32229.

22. Najjar et al., 1993, J. Biol. Chem. 268: 1201-1206.

23. Nédellec et al., 1994, J. Virol. 68: 4525-4537.

24. Nédellec et al., 1995, Eur. J. Biochem. 231: 104-114.

25. Öbrink, B., 1997, Curr. Opin. Cell Biol. 9: 616-626.

26. Prall et al., 1996, J. Histochem. & Cytochem. 44: 35-41.

27. Racaniello et al., 1993, Dev. Biol. Stand. 78:109-116.

28. Rao et al., 1997, Virology. 229:336-348. 29. Rao et al., 1998, Adv Exp Med Biol 440:549-555.

30. Smith et al., 1991, J. Infect. Dis. 163:879-882.

31. Thomas et al., 1987, Cell 51: 503-512.

32. Wessner et al., 1998, J. Virol. 72:1941-1948.

33. Zelus et al., 1998, J. Virol. 72:7237-7244.

34. Zimmermann, W., 1998, The nature and expression of the rodent CEA families: evolutionary considerations. P. 31-56. In Stanners, C. P. (ed.), Cell Adhesion and Communication Mediated by the CEA Family. Harwood Academic Publishers, Amsterdam.

35. Leusch et al. 1991. Infection & Immunity: 59(4):2051-7.

36. Boulton et al., Neisserial binding to CEACAM1 arrests the activation and proliferation of CD4+ T lymphocytes. Nat Immunol. 2002 Feb. 19 [epub ahead of print].

37. Nakajima et al., J. Immunol. 2002 Feb. 1;168(3):1028-35.

38. Chen et al., J Leukoc Biol. 2001 August;70(2):335-40.

39. Kammerer et al., J. Immunol. 2001 Jun. 1;166(11):6537-44.

40. Morales et al., J. Immunol. 1999 Aug. 1; 163(3):1363-70.

1. A transgenic non human animal comprising a disruption of the Ceacam1 gene wherein said animal expresses a reduced level of CEACAM1 relative to a corresponding wild-type animal.

2. The transgenic non-human animal as recited in of claim 1, wherein said animal is a mouse.

3. The transgenic mouse of claim 2, wherein said Ceacam1 gene is the Ceacam1^{a} allele.

4. The transgenic mouse of claim 2, wherein said disruption is a homozygous disruption.

5. The transgenic mouse of claim 3, wherein said disruption results from an insertion of a positive selection expression cassette into the Ceacam1 gene.

6. The transgenic mouse of claim 5, wherein the positive selection expression cassette is inserted into intron 2 of the Ceacam1 gene.

7. The transgenic mouse of claim 6 wherein said disruption modulates the level of alternative splicing of the Ceacam1 gene.

8. A method for producing a transgenic non human animal wherein said animal expresses a reduced level of CEACAM1 relative to a corresponding wild-type animal, comprising:
(a) introducing a CEACAM1 targeting vector into an embryonic stem cell of said non human animal;
(b) introducing said embryonic stem cell of a) into a blastoctocyst of said non human animal;
(c) transplanting said blastocyst into a pseudopregnant of said non-human animal;
(d) allowing said blastocyst to develop to term;
(e) identifying a transgenic non-human animal whose genome comprises a disruption of the Ceacam1 gene in at least one allele;
(f) breeding the transgenic animal of step (e) to obtain an offspring of said transgenic animal whose genome comprises a disruption of the Ceacam1,
thereby producing a transgenic non-human animal wherein said disruption results in decreased levels of CEACAM1 relative to a wild-type form of said non-human animal.

9. The method of claim 8, wherein said non human animal is a mouse.

10. The method of claim 8, wherein said disruption is homozygous.

11. The method of claim 9, wherein said Ceacam1 is the Ceacam1^{a}.

12. The method of claim 11, wherein said Ceacam1 targeting vector comprises a positive expression cassette.

13. The method of claim 12, wherein said positive expression cassette comprises a neo gene operatively linked to at least one regulatory element.

14. The method of claim 13, wherein said positive expression cassette is inserted into an intron of the Ceacam1 gene.

15. The method of claim 14, wherein said intron is intron 2.

16. A method of modulating infection by an infectious agent which utilizes CEACAM1 as a cellular receptor comprising modulating of the expression of Ceacam1 so that the ratio of 4 Ig isoforms of CEACAM1 over 2 Ig isoforms of CEACAM1 is reduced relative to a corresponding wild-type cell.

17. The method of claim 16 wherein alternative splicing of the Ceacam1 gene is affected.

18. The method of claims 16, wherein said infectious agent is MHV, Salmonella or E. coli.

19. The method a of claim 9, wherein said transgenic mouse expresses a decreased ratio of Ceacam14 Ig/Ceacam12 Ig relative to a corresponding wild-type mouse comprising.

20. The method in claim 19, wherein said disruption is homozygous.

21. A method of identifying a compound able to modulate MHV infection sensitivity comprising:
(a) applying a candidate compound to cells;
(b) determining the expressed ratio of isoforms 4 Ig CEACAM1 over isoforms 2 Ig CEACAM1 relative to a control cell,
whereby a compound which modulates MHV infection sensitivity is selected when said ratio in the cell treated with said candidate compound is measurably different from that of an untreated cell.

22. A method of modulating MHV infection sensitivity in a mouse comprising a modulating the expression of Ceacam1 so that the ratio of 4 Ig isoforms of CEACAM1 over 2 Ig isoforms of CEACAM1 is reduced relative to that of a corresponding wild-type mouse.

23. A method of modulating MHV infection sensitivity in a mouse comprising modulating the expression of Ceacam1 so that the total amount of CEACAM1 isoforms expressed is reduced relative to that of a corresponding wild-type mouse.
